(19) United States
(12) Patent Application Publication
(10) Pub. No.: US 2006/0276539 A1
(43) Pub. Date: Dec. 7, 2006

(54) Treating Androgen Decline in Aging Male (ADAM)- associated conditions with SARMS

(76) Inventors: James T. Dalton, Upper Arlington, OH (US); Duane D. Miller, Germantown, TN (US); Mitchell S. Steiner, Germantown, TN (US); Karen A. Veverka, Cordova, TN (US)

Correspondence Address: PEARL COHEN ZEDEK, LLP, 1500 BROADWAY 12TH FLOOR, NEW YORK, NY, 10036, US

(21) Appl. No.: 11/324,753

(22) Filed: Jan. 4, 2006

Related U.S. Application Data
(63) Continuation-in-part of application No. 10/683,161, filed on Oct. 14, 2003.
(60) Provisional application No. 60/418,336, filed on Oct. 16, 2002.

(51) Int. Cl. A61K 31/277

(52) U.S. Cl. 514/521

(57) ABSTRACT

This invention provides methods for treatment and inhibition of a male subject having an Androgen Decline in Aging Male (ADAM)-associated condition, for example sexual dysfunction, decreased sexual libido, erectile dysfunction, hypogonadism, sarcopenia, osteopenia, osteoporosis, an alteration in cognition and mood, depression, anemia, hair loss, obesity, muscle loss, dry eye, memory loss, benign prostate hyperplasia and/or prostate cancer, by administering to the subject a selective androgen receptor modulator (SARM) compound and/or its analog, derivative, isomer, metabolite, pharmaceutically acceptable salt, pharmaceutical product, hydrate, N-oxide, pro-drug, polymorph, crystal, or any combination thereof.

### CROSS REFERENCE TO RELATED APPLICATIONS

This application is a Continuation-in-Part of U.S. application Ser. No. 10/683,161, filed Oct. 14, 2003, which claims the benefit of U.S. Provisional Application Ser. No. 60/418,336, filed Oct. 16, 2002, which is incorporated in its entirety by reference herein.

### FIELD OF INVENTION

This invention provides methods for treatment and inhibition of conditions associated with Androgen Decline in Aging Males (ADAM). The conditions include inter alia sexual dysfunction, decreased sexual libido, erectile dysfunction, hypogonadism, sarcopenia, osteopenia, osteoporosis, an alteration in cognition and mood, depression, anemia, hair loss, obesity, muscle loss, dry eye, memory loss, benign prostate hyperplasia and/or prostate cancer. The methods include administering to the subject a selective androgen receptor modulator (SARM) compound and/or its analog, derivative, isomer, metabolite, pharmaceutically acceptable salt, pharmaceutical product, hydrate, N-oxide, pro-drug, polymorph, crystal, or any combination thereof.

### BACKGROUND OF THE INVENTION

“ADAM” refers to a progressive decrease in androgen production, common in males after middle age. The syndrome is characterized by alterations in the physical and intellectual domains that correlate with and can be corrected by manipulation of the androgen milieu.

ADAM is characterized biochemically by a decrease not only in serum androgen, but also in other hormones, such as growth hormone, melatonin and dehydroepiandrosterone. Clinical manifestations of ADAM include fatigue, depression, decreased libido, sexual dysfunction, erectile dysfunction, sarcopenia, osteopenia, osteoporosis, benign prostate hyperplasia (BPH), hypogonadism, alterations in mood and cognition, depression, anemia, obesity, muscle loss, dry eye, memory loss, hair loss and prostate cancer.

Innovative approaches are urgently needed at both the basic science and clinical levels to treat ADAM. The present invention is directed to satisfying this need.

### SUMMARY OF THE INVENTION

The present invention provides methods of treating, suppressing, inhibiting or reducing the incidence of an Androgen Decline in Aging Male (ADAM)-associated condition in a male subject, by administering to the subject a selective androgen receptor modulator (SARM) compound and/or its analog, derivative, isomer, metabolite, pharmaceutically acceptable salt, pharmaceutical product, hydrate, N-oxide, pro-drug, polymorph, crystal, or any combination thereof. The present invention provides methods of treating, preventing, suppressing, inhibiting or reducing the incidence of, inter alia, sexual dysfunction, decreased sexual libido, erectile dysfunction, hypogonadism, sarcopenia, osteopenia, osteoporosis, an alteration in cognition and mood, depression, anemia, hair loss, obesity, muscle loss, BPH, dry eye, memory loss, or prostate cancer due to ADAM in a male subject, by administering to the subject a SARM compound and/or its analog, derivative, isomer, metabolite, pharmaceutically acceptable salt, pharmaceutical product, hydrate, N-oxide, pro-drug, polymorpli, crystal, or any combination thereof.

Thus, in one embodiment, the present invention provides a method of treating a male subject having an ADAM-associated condition, the method comprising administering to the subject a SARM compound represented by a structure of formula VIII: 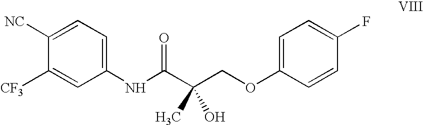
or an analog, derivative, isomer, metabolite, pharmaceutically acceptable salt, pharmaceutical product, hydrate or N-oxide, pro-drug, polymorph or crystal thereof, thereby treating a male subject having an ADAM-associated condition.

In another embodiment, the present invention provides a method of treating a male subject having an ADAM-associated condition, the method comprising administering to the subject a SARM compound represented by a structure of formula IX: 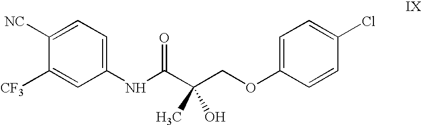
or an analog, derivative, isomer, metabolite, pharmaceutically acceptable salt, pharmaceutical product, hydrate or N-oxide, pro-drug, polymorph or crystal thereof, thereby treating a male subject having an ADAM-associated condition.

In another embodiment, the present invention provides a method of treating a male subject having an ADAM-associated condition, the method comprising administering to the subject a SARM compound represented by a structure of formula X: 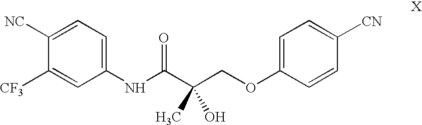
or an analog, derivative, isomer, metabolite, pharmaceutically acceptable salt, pharmaceutical product, hydrate or N-oxide, pro-drug, polymorph or crystal thereof, thereby treating a male subject having an ADAM-associated condition.

In another embodiment, the present invention provides a method of treating a male subject having an ADAM-associated condition, the method comprising administering to the subject a SARM compound represented by a structure of formula XI: 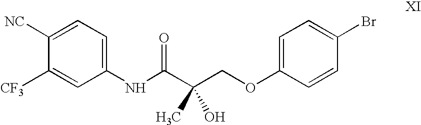
or an analog, derivative, isomer, metabolite, pharmaceutically acceptable salt, pharmaceutical product, hydrate or N-oxide, pro-drug, polymorph or crystal thereof, thereby treating a male subject having an ADAM-associated condition.

In another embodiment, the present invention provides a method of suppressing, inhibiting or reducing an incidence of an ADAM-associated condition in a male subject, the method comprising administering to the subject a SARM compound represented by a structure of formula VIII: 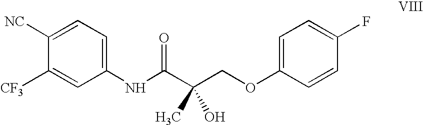
or an analog, derivative, isomer, metabolite, pharmaceutically acceptable salt, pharmaceutical product, hydrate or N-oxide, pro-drug, polymorph or crystal thereof, thereby suppressing, inhibiting or reducing an incidence of an ADAM-associated condition in a male subject.

In another embodiment, the present invention provides a method of suppressing, inhibiting or reducing an incidence of an ADAM-associated condition in a male subject, the method comprising administering to the subject a SARM compound represented by a structure of formula IX: 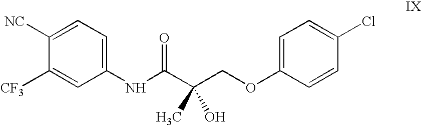
or an analog, derivative, isomer, metabolite, pharmaceutically acceptable salt, pharmaceutical product, hydrate or N-oxide, pro-drug, polymorph or crystal thereof, thereby suppressing, inhibiting or reducing an incidence of an ADAM-associated condition in a male subject.

In another embodiment, the present invention provides a method of suppressing, inhibiting or reducing an incidence of an ADAM-associated condition in a male subject, the method comprising administering to the subject a SARM compound represented by a structure of formula X: 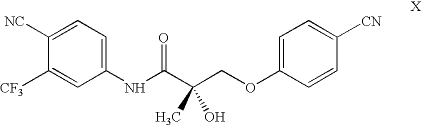
or an analog, derivative, isomer, metabolite, pharmaceutically acceptable salt, pharmaceutical product, hydrate or N-oxide, pro-drug, polymorph or crystal thereof, thereby suppressing, inhibiting or reducing an incidence of an ADAM-associated condition in a male subject.

In another embodiment, the present invention provides a method of suppressing, inhibiting or reducing an incidence of an ADAM-associated condition in a male subject, the method comprising administering to the subject a SARM compound represented by a structure of formula XI: 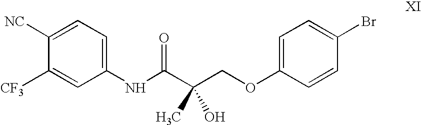
or an analog, derivative, isomer, metabolite, pharmaceutically acceptable salt, pharmaceutical product, hydrate or N-oxide, pro-drug, polymorph or crystal thereof, thereby suppressing, inhibiting or reducing an incidence of an ADAM-associated condition in a male subject.

In another embodiment, the present invention provides a method of treating a male subject having a sexual dysfunction, decreased sexual libido, erectile dysfunction, hypogonadism, sarcopenia, osteopenia, osteoporosis, an alteration in cognition and mood, depression, anemia, hair loss, obesity, muscle loss, BPH, dry eye, memory loss, or prostate cancer due ADAM, the method comprising administering to the subject a SARM compound represented by a structure of formula VIII: 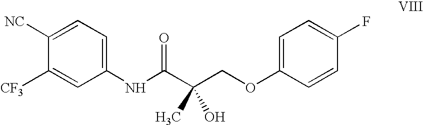
or an analog, derivative, isomer, metabolite, pharmaceutically acceptable salt, pharmaceutical product, hydrate or N-oxide, pro-drug, polymorph or crystal thereof, thereby treating a male subject having a sexual dysfunction, decreased sexual libido, erectile dysfunction, hypogonadism, sarcopenia, osteopenia, osteoporosis, an alteration in cognition and mood, depression, anemia, hair loss, obesity, muscle loss, BPH, dry eye, memory loss, or prostate cancer due ADAM.

In another embodiment, the present invention provides a method of treating a male subject having a sexual dysfunction, decreased sexual libido, erectile dysfunction, lypogonadism, sarcopenia, osteopenia, osteoporosis, an alteration in cognition and mood, depression, anemia, hair loss, obesity, muscle loss, BPH, dry eye, memory loss, or prostate cancer due ADAM, the method comprising administering to the subject a SARM compound represented by a structure of formula IX: 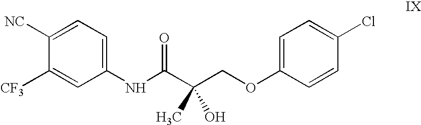
or an analog, derivative, isomer, metabolite, pharmaceutically acceptable salt, pharmaceutical product, hydrate or N-oxide, pro-drug, polymorph or crystal thereof, thereby treating a male subject having a sexual dysfunction, decreased sexual libido, erectile dysfunction, hypogonadism, sarcopenia, osteopenia, osteoporosis, an alteration in cognition and mood, depression, anemia, hair loss, obesity, muscle loss, BPH, dry eye, memory loss, or prostate cancer due ADAM.

In another embodiment, the present invention provides a method of treating a male subject having a sexual dysfunction, decreased sexual libido, erectile dysfunction, hypogonadism, sarcopenia, osteopenia, osteoporosis, an alteration in cognition and mood, depression, anemia, hair loss, obesity, muscle loss, BPH, dry eye, memory loss, or prostate cancer due ADAM, the method comprising administering to the subject a SARM compound represented by a structure of formula X: 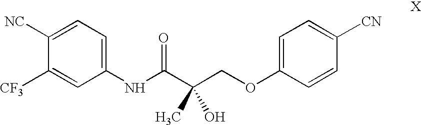
or an analog, derivative, isomer, metabolite, pharmaceutically acceptable salt, pharmaceutical product, hydrate or N-oxide, pro-drug, polymorph or crystal thereof, thereby treating a male subject having a sexual dysfunction, decreased sexual libido, erectile dysfunction, hypogonadism, sarcopenia, osteopenia, osteoporosis, an alteration in cognition and mood, depression, anemia, hair loss, obesity, muscle loss, BPH, dry eye, memory loss, or prostate cancer due ADAM.

In another embodiment, the present invention provides a method of treating a male subject having a sexual dysfunction, decreased sexual libido, erectile dysfunction, hypogonadism, sarcopenia, osteopenia, osteoporosis, an alteration in cognition and mood, depression, anemia, hair loss, obesity, muscle loss, BPH, dry eye, memory loss, or prostate cancer due ADAM, the method comprising administering to the subject a SARM compound represented by a structure of formula XI: 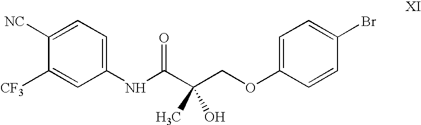
or an analog, derivative, isomer, metabolite, pharmaceutically acceptable salt, pharmaceutical product, hydrate or N-oxide, pro-drug, polymorph or crystal thereof, thereby treating a male subject having a sexual dysfunction, decreased sexual libido, erectile dysfunction, hypogonadism, sarcopenia, osteopenia, osteoporosis, an alteration in cognition and mood, depression, anemia, hair loss, obesity, muscle loss, BPH, dry eye, memory loss, or prostate cancer due ADAM.

### BRIEF DESCRIPTION OF THE DRAWINGS

FIG. 1: Flowchart of ADAM-associated conditions.

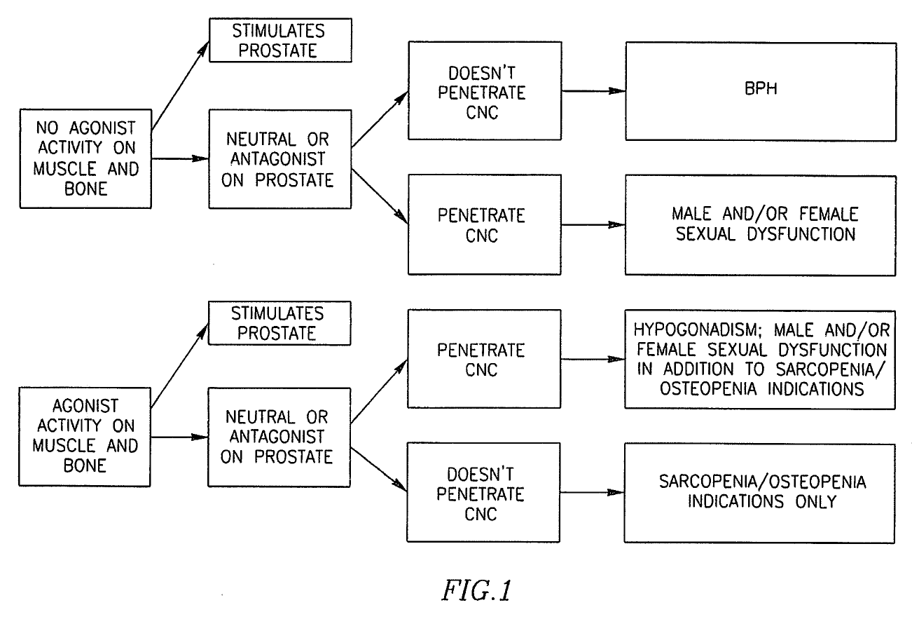

FIG. 2: Schematic illustration of ADAM-associated conditions.

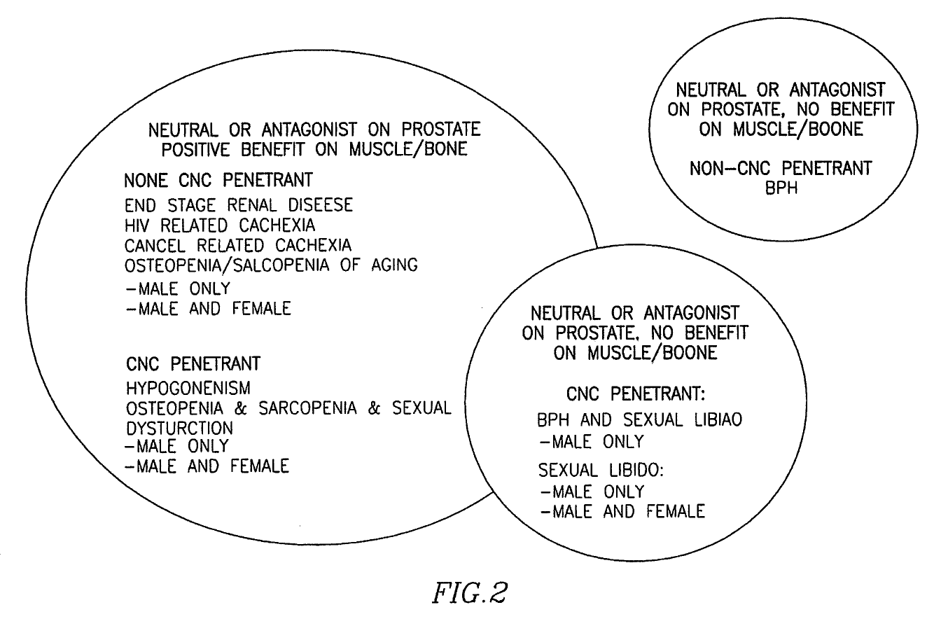

FIG. 3: Androgenic and anabolic activity of Compound VI and Compound VII in rats. Male rats with normal testicular function (no surgical manipulation) were left untreated (Intact), treated with compound VI (0.5 mg/day), compound VII (0.5 milligram (mg)/day) or testosterone proprionate (TP, 0.5 mg/day), and the weight of androgen-responsive tissues (prostate—FIG. 3A, seminal vesicles—FIG. 3B, and levator ani muscle—FIG. 3C) was determined,

FIG. 4: Androgenic and anabolic activity of Compound VI and Compound VII in rats. Male rats received unilateral orchidectomy (Hemi-orchidectomized) and were left untreated (Intact), treated with vehicle alone (PEG 300), Compound VI (0.5 mg/day), Compound VII (0.5 mg/day), or testosterone proprionate (TP, 0.5 mg/day), and the weight of androgen-responsive tissues (prostate—FIG. 4A, seminal vesicles—FIG. 4B, and levator ani muscle—FIG. 4C) was determined.

FIG. 5: Androgenic and anabolic activity of Compound VI and Compound VII in rats. Male rats received bilateral orclidectomy (Castrated) and were left untreated (Intact), treated with vehicle alone (PEG 300), Compound VI (0.5 mg/day), Compound VII (0.5 mg/day), or testosterone proprionate (TP, 0.5 mg/day), and the weight of androgen-responsive tissues (prostate—FIG. 5A, seminal vesicles—FIG. 5B, and levator ani muscle—FIG. 5C) was determined.

FIG. 6: Dose response Curves. Rats were left untreated, or treated with 0.1, 0.3, 0.5, 0.75 and 1.0 mg/day Compound VI, Compound VII or testosterone propionate (TP), and the weight of androgen-responsive tissues (prostate—FIG. 6A, seminal vesicles—FIG. 6B and levator ani muscle—FIG. 6C) was determined. The results are plotted as percentage of the intact control.

FIG. 7: Effect of testosterone proprionate and Compound VI on myosin heavy chain (MHC) IIb mRNA expression. FIG. 7A: histogram showing effect of Compound VI on MHC IIb mRNA expression. FIG. 7B: raw RT-PCR data, showing MHC IIb mRNA expression.

FIG. 8: Effect of SARMS on Bone Mineral Content (BMC) and Bone Mineral Density (BMD) in female rates after ovariectomy.

FIG. 9: Left panel: Compound VI increased whole body BMC after 120 days. Right panel: time course of BMC changes in response to 3 milligrams/day Compound VI treatment.

FIG. 10: Compound VI exerted a protective effect at both the L2-L4 vertebra (left panel) and proximal femur (right panel).

FIG. 11: Compound VI increased biomechanical strength of the L5 vertebra (left panel) and femur (right panel).

FIG. 12: Compound VI increased cortical thickness (left panel) and trabecular density (right panel) in the femoral mid-shaft.

### DETAILED DESCRIPTION OF THE INVENTION

The present invention provides methods of treating, suppressing, inhibiting or reducing the incidence of an ADAM-associated condition in a male subject, by administering to the subject a SARM compound and/or its analog, derivative, isomer, metabolite, pharmaceutically acceptable salt, pharmaceutical product, hydrate, N-oxide, pro-drug, polymorph, crystal, or any combination thereof. The present invention further provides methods of treating, preventing, suppressing, inhibiting or reducing the incidence of sexual dysfunction, decreased sexual libido, erectile dysfunction, hypogonadism, sarcopenia, osteopenia, osteoporosis, an alteration in cognition and mood, depression, anemia, hair loss, obesity, muscle loss, BPH, dry eye, memory loss, and/or prostate cancer due to ADAM in a male subject, by administering to the subject a SARM compound and/or its analog, derivative, isomer, metabolite, pharmaceutically acceptable salt, pharmaceutical product, hydrate, N-oxide, pro-drug, polymorph, crystal, or any combination thereof.

Thus, in one embodiment, the present invention provides a method of treating a male subject having an ADAM-associated condition, the method comprising administering to the subject a SARM compound represented by a structure of formula VIII: 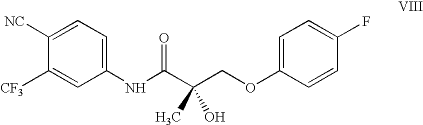
or an analog, derivative, isomer, metabolite, pharmaceutically acceptable salt, pharmaceutical product, hydrate or N-oxide, pro-drug, polymorph or crystal thereof, thereby treating a male subject having an ADAM-associated condition.

In another embodiment, the present invention provides a method of treating a male subject having an ADAM-associated condition, the method comprising administering to the subject a SARM compound represented by a structure of formula IX: 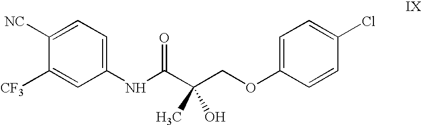
or an analog, derivative, isomer, metabolite, pharmaceutically acceptable salt, pharmaceutical product, hydrate or N-oxide, pro-drug, polymorph or crystal thereof, thereby treating a male subject having an ADAM-associated condition.

In another embodiment, the present invention provides a method of treating a male subject having an ADAM-associated condition, the method comprising administering to the subject a SARM compound represented by a structure of formula X: 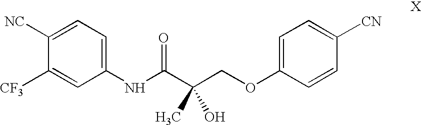
or an analog, derivative, isomer, metabolite, pharmaceutically acceptable salt, pharmaceutical product, hydrate or N-oxide, pro-drug, polymorph or crystal thereof, thereby treating a male subject having an ADAM-associated condition.

In another embodiment, the present invention provides a method of treating a male subject having an ADAM-associated condition, the method comprising administering to the subject a SARM compound represented by a structure of formula XI: 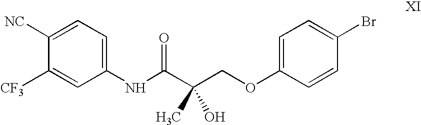
or an analog, derivative, isomer, metabolite, pharmaceutically acceptable salt, pharmaceutical product, hydrate or N-oxide, pro-drug, polymorph or crystal thereof, thereby treating a male subject having an ADAM-associated condition.

For example, the findings described in Example 1 show that SARM compounds VI and VII are useful in treating ADAM-associated conditions, as evidenced by their androgenic and/or anabolic activity. “Androgenic activity” refers, in one embodiment, to androgen receptor (AR) agonist activity in androgenic target tissues, such as prostate and seminal vesicles. Androgenic effects were shown by increases in weights of the prostate and seminal vesicles, which are accepted in the art as indicators of androgenic activity (Almeida S A et al, Braz J Med Biol Res 31(11): 1443-8, November 1998; Lemus A E et al, J Steroid Biochem Mol Biol 60(1-2): 121-9, January 1997).

The AR is a ligand-activated transcriptional regulatory protein that mediates induction of male sexual development and function through its activity with endogenous androgens (male sex hormones). The androgenic hormones are steroids that are produced in the body by the testis and the cortex of the adrenal gland. Androgenic steroids play an important role in many physiologic processes, including the development and maintenance of male sexual characteristics such as muscle and bone mass, prostate growth, spermatogenesis, and the male hair pattern (Matsumoto, Endocrinol. Met. Clin. N. Am. 23:857-75 (1994)). The endogenous steroidal androgens include testosterone and dihydrotestosterone (“DHT”). Other steroidal androgens include esters of testosterone, such as the cypionate, propionate, phenylpropionate, cyclopentylpropionate, isocarporate, enanthate, and decanoate esters, and other synthetic androgens such as 7-Methyl-Nortestosterone (“MENT′”) and its acetate ester (Sundaram et al., “7 Alpha-Methyl-Nortestosterone(MENT): The Optimal Androgen For Male Contraception,” Ann. Med., 25:199-205 (1993) (“Sundaram”)).

A receptor agonist is, in one embodiment, a substance that binds a receptor and activates it. A receptor partial agonist is, in one embodiment, a substance that binds a receptor and partially activates it. A receptor antagonist is, in one embodiment, a substance that binds a receptor and inactivates it. As demonstrated herein, the SARM compounds of the present invention have a tissue-selective effect, wherein one agent may be an agonist, partial agonist and/or antagonist, depending on the tissue. For example, the SARM compound may stimulate muscle tissue and at the same time inhibit prostate tissue. In one embodiment, the SARM compound is an AR agonist, and is, therefore, useful in binding to and activating the AR. In another embodiment, the SARM compound is an AR antagonist, and is, therefore, useful in binding to and inactivating the AR. Assays to determine whether the compounds of the present invention are AR agonists or antagonists are well known to a person skilled in the art. For example, AR agonistic activity can be determined by monitoring the ability of the SARM compounds to maintain and/or stimulate the growth of AR containing tissue such as prostate and seminal vesicles, as measured by weight. AR antagonistic activity can be determined by monitoring the ability of the SARM compounds inhibit the growth of AR containing tissue.

In another embodiment, a SARM compound of the present invention can be classified as a partial AR agonist/antagonist. In this embodiment, a SARM compound is an AR agonist in some tissues, causing increased transcription of AR-responsive genes (e.g. muscle anabolic effect). In other tissues, the compound serves as a competitive inhibitor of testosterone/DHT on the AR, preventing agonistic effects of native androgens.

In one embodiment, the SARM compound of the present invention binds reversibly to the AR. In another embodiment, the SARM compound binds irreversibly to the AR. The compounds of the present invention may contain a functional group (affinity label) that allows alkylation of the AR (i.e. covalent bond formation). Thus, in this case, the compound binds irreversibly to the receptor and, accordingly, cannot be displaced by a steroid, such as the endogenous ligands DHT and testosterone.

“Anabolic activity” refers, in one embodiment, to increasing the mass of a connective tissue. In another embodiment, “anabolic activity” refers to increasing the strength of a connective tissue. In one embodiment, the connective tissue is cortical bone. In another embodiment, the connective tissue is trabecular bone. In another embodiment, the connective tissue is cancellous bone. In another embodiment, the connective tissue is muscle. In another embodiment, the connective tissue is cartilage. In another embodiment, the connective tissue is any other type of connective tissue known in the art Increases in the weight of the levator ani muscle were used in the present invention to demonstrate anabolic activity, and are accepted in the art as a reliable index of anabolic activity (Antonio J et al, “Effects of castration and androgen treatment on androgen-receptor levels in rat skeletal muscles,” J Appl Physiol 87: 2016-2019, 1999). Anabolic activity in bone and muscle synergize, in one embodiment, to decrease fracture rates in a subject.

In another embodiment, anabolic activity is a manifestation of AR agonistic activity in a connective tissue. Each type of anabolic activity represents a separate embodiment of the present invention.

In another embodiment, the present invention provides a method of suppressing, inhibiting or reducing an incidence of an ADAM-associated condition in a male subject, the method comprising administering to the subject a SARM compound represented by a structure of formula VIII: 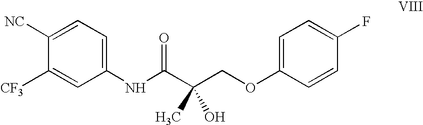
or an analog, derivative, isomer, metabolite, pharmaceutically acceptable salt, pharmaceutical product, hydrate or N-oxide, pro-drug, polymorph or crystal thereof, thereby suppressing, inhibiting or reducing an incidence of an ADAM-associated condition in a male subject.

In another embodiment, the present invention provides a method of suppressing, inhibiting or reducing an incidence of an ADAM-associated condition in a male subject, the method comprising administering to the subject a SARM compound represented by a structure of formula IX: 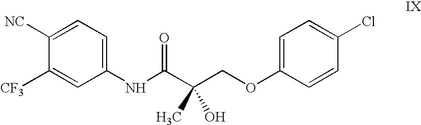
or an analog, derivative, isomer, metabolite, pharmaceutically acceptable salt, pharmaceutical product, hydrate or N-oxide, pro-drug, polymorph or crystal thereof, thereby suppressing, inhibiting or reducing an incidence of an ADAM-associated condition in a male subject.

In another embodiment, the present invention provides a method of suppressing, inhibiting or reducing an incidence of an ADAM-associated condition in a male subject, the method comprising administering to the subject a SARM compound represented by a structure of formula X: 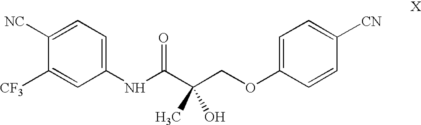
or an analog, derivative, isomer, metabolite, pharmaceutically acceptable salt, pharmaceutical product, hydrate or N-oxide, pro-drug, polymorph or crystal thereof, thereby suppressing, inhibiting or reducing an incidence of an ADAM-associated condition in a male subject.

In another embodiment, the present invention provides a method of suppressing, inhibiting or reducing an incidence of an ADAM-associated condition in a male subject, the method comprising administering to the subject a SARM compound represented by a structure of formula XI: 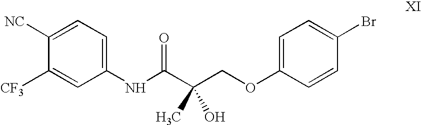
or an analog, derivative, isomer, metabolite, pharmaceutically acceptable salt, pharmaceutical product, hydrate or N-oxide, pro-drug, polymorph or crystal thereof, thereby suppressing, inhibiting or reducing an incidence of an ADAM-associated condition in a male subject.

In another embodiment, the present invention provides a method of treating a male subject having a sexual dysfunction, decreased sexual libido, erectile dysfunction, hypogonadism, sarcopenia, osteopenia, osteoporosis, an alteration in cognition and mood, depression, anemia, hair loss, obesity, muscle loss, BPH, dry eye, memory loss, or prostate cancer due ADAM, the method comprising administering to the subject a SARM compound represented by a structure of formula VIII: 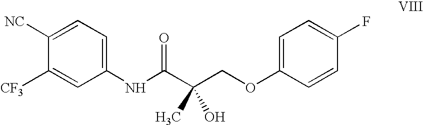
or an analog, derivative, isomer, metabolite, pharmaceutically acceptable salt, pharmaceutical product, hydrate or N-oxide, pro-drug, polymorph or crystal thereof, thereby treating a male subject having a sexual dysfunction, decreased sexual libido, erectile dysfunction, hypogonadism, sarcopenia, osteopenia, osteoporosis, an alteration in cognition and mood, depression, anemia, hair loss, obesity, muscle loss, BPH, dry eye, memory loss, or prostate cancer due ADAM.

In another embodiment, the present invention provides a method of treating a male subject having a sexual dysfunction, decreased sexual libido, erectile dysfunction, hypogonadism, sarcopenia, osteopenia, osteoporosis, an alteration in cognition and mood, depression, anemia, hair loss, obesity, muscle loss, BPH, dry eye, memory loss, or prostate cancer due ADAM, the method comprising administering to the subject a SARM compound represented by a structure of formula IX: 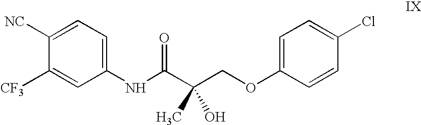
or an analog, derivative, isomer, metabolite, pharmaceutically acceptable salt, pharmaceutical product, hydrate or N-oxide, pro-drug, polymorph or crystal thereof, thereby treating a male subject having a sexual dysfunction, decreased sexual libido, erectile dysfunction, hypogonadism, sarcopenia, osteopenia, osteoporosis, an alteration in cognition and mood, depression, anemia, hair loss, obesity, muscle loss, BPH, dry eye, memory loss, or prostate cancer due ADAM.

In another embodiment, the present invention provides a method of treating a male subject having a sexual dysfunction, decreased sexual libido, erectile dysfunction, hypogonadism, sarcopenia, osteopenia, osteoporosis, an alteration in cognition and mood, depression, anemia, hair loss, obesity, muscle loss, BPH, dry eye, memory loss, or prostate cancer due ADAM, the method comprising administering to the subject a SARM compound represented by a structure of formula X: 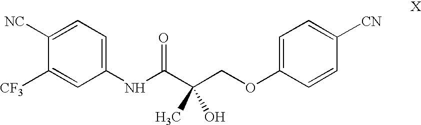
or an analog, derivative, isomer, metabolite, pharmaceutically acceptable salt, pharmaceutical product, hydrate or N-oxide, pro-drug, polymorph or crystal thereof, thereby treating a male subject having a sexual dysfunction, decreased sexual libido, erectile dysfunction, hypogonadism, sarcopenia, osteopenia, osteoporosis, an alteration in cognition and mood, depression, anemia, hair loss, obesity, muscle loss, BPH, dry eye, memory loss, or prostate cancer due ADAM.

In another embodiment, the present invention provides a method of treating a male subject having a sexual dysfunction, decreased sexual libido, erectile dysfunction, hypogonadism, sarcopenia, osteopenia, osteoporosis, an alteration in cognition and mood, depression, anemia, hair loss, obesity, muscle loss, BPH, dry eye, memory loss, or prostate cancer due ADAM, the method comprising administering to the subject a SARM compound represented by a structure of formula XI: 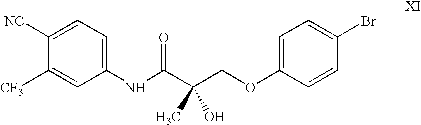
or an analog, derivative, isomer, metabolite, pharmaceutically acceptable salt, pharmaceutical product, hydrate or N-oxide, pro-drug, polymorph or crystal thereof, thereby treating a male subject having a sexual dysfunction, decreased sexual libido, erectile dysfunction, hypogonadism, sarcopenia, osteopenia, osteoporosis, an alteration in cognition and mood, depression, anemia, hair loss, obesity, muscle loss, BPH, dry eye, memory loss, or prostate cancer due ADAM.

For example, the findings described in Example 1 show that SARM compounds VI and VII exhibit full agonist or mixed-agonist AR activity, depending on the target tissue, and that the degree of activity and tissue-specificity varies between different SARM compounds. Thus, particular SARM compounds can be chosen to treat particular ADAM-associated conditions, depending on the degree and type (agonist, mixed agonist, or antagonist) of AR activity required and the target tissues of importance.

In one embodiment, an anabolic activity of SARM compound is used in treating an ADAM-associated condition that is remediated by anabolic activity. In one embodiment, the condition is osteopenia. In another embodiment, the condition is osteoporosis. In another embodiment, the condition is obesity. In another embodiment, the condition is sarcopenia. The present invention (Examples 2 and 3) shows that SARM compounds are anabolic in both cortical and trabecular bone and muscle in testosterone-depleted subjects, and subjects with slight androgen depletion. In addition, SARM compounds were demonstrated to prevent bone resorption in response to androgen deprivation. Thus, SARM compounds have utility in (a) treating ADAM-associated conditions that are remediated by anabolic activity (e.g. reversing connective tissue loss); and (b) inhibiting or reducing the incidence of such ADAM-associated conditions (e.g. preventing connective tissue loss due to anticipated androgen deprivation.

Thus, in one embodiment, the subject of the present invention is androgen-depleted. In another embodiment, the subject is androgen deficient. In another embodiment, the subject has normal levels of androgen. In another embodiment, the subject will soon undergo a treatment that will deplete his androgen levels. In another embodiment, the subject is an aging male subject. Each possibility represents a separate embodiment of the present invention.

In the present invention, SARM compounds were shown to have no effect on levels of testosterone, FSH, and LH. Thus, in one embodiment, the subject of the present invention is a subject in which therapy that affects testosterone, FSH, or LH levels is contra-indicated. In one embodiment, the subject for whom such therapy is contra-indicated is a subject at risk for anosmia, visual abnormalities, or headaches. In another embodiment, the subject or his/her physician wishes to avoid affecting levels of testosterone, FSH, and LH to avoid aggravating an ADAM-associated condition.

In another embodiment, the male subject of the present invention is an aging male subject. In one embodiment, the term “aging” means a process of becoming older. In one embodiment, the aging male is a male over 40 years old. In another embodiment, the aging male is a male over 45 years old. In another embodiment, the aging male is a male over 45 years old. In another embodiment, the aging male is a male over 50 years old. In another embodiment, the aging male is a male over 55 years old. In another embodiment, the aging male is a male over 60 years old. In another embodiment, the aging male is a male over 65 years old. In another embodiment, the aging male is a male over 70 years old. In another embodiment, the aging male is a male over 75 years old.

In another embodiment, an androgenic activity of a SARM compound is used to treat an ADAM-related condition. In one embodiment, the condition is sexual dysfunction. In another embodiment, the condition is osteoporosis. In another embodiment, the condition is obesity. In another embodiment, the condition is sarcopenia. In another embodiment, the condition is decreased sexual libido. In another embodiment, the condition is erectile dysfunction. In another embodiment, the condition is hypogonadism. In another embodiment, the condition is an alteration in cognition and mood. In another embodiment, the condition is depression. In another embodiment, the condition is BPH. In another embodiment, the condition is anemia. In another embodiment the condition is muscle loss. In another embodiment the condition is dry eye. In another embodiment the condition is memory loss.

The present invention shows that SARM compounds exhibit AR mixed agonist activity in androgenic target tissues such as prostate and seminal vesicles. Since the level and type of AR activity and the affected tissues vary between different SARM compounds, it is possible to choose the appropriate SARM compound based on the type of AR activity and the target tissue desired.

Thus, in another embodiment, an antagonistic AR activity of a SARM compound in an androgenic target tissue is used to treat an ADAM-related condition. In one embodiment, the condition is BPH. In another embodiment, the condition is hair loss. In another embodiment, the condition is prostate cancer.

In one embodiment, the ADAM-associated condition is sexual dysfunction. In one embodiment, the sexual dysfunction is a desire disorders. In another embodiment, the sexual dysfunction is an arousal disorders. In another embodiment, the sexual dysfunction is an orgasm disorder. In another embodiment, the sexual dysfunction is a pain disorder. Each type of sexual dysfunction represents a separate embodiment of the present invention.

In another embodiment, the ADAM-associated condition is decreased sexual libido. The term “libido, in one embodiment, means sexual desire.

In another embodiment, the ADAM-associated condition is erectile dysfunction. The term “erectile,” in one embodiment, means capable of being erected. An erectile tissue is a tissue which is capable of being greatly dilated and made rigid by the distension of the numerous blood vessels which it contains.

In another embodiment, the ADAM-associated condition is hypogonadism. “Hypogonadism,” in one embodiment, is a condition resulting from or characterised by abnormally decreased functional activity of the gonads, with retardation of growth and sexual development.

In another embodiment, the ADAM-associated condition is sarcopenia. In one embodiment, the sarcopenia comprises muscle loss. In another embodiment, the sarcopenia comprising weight loss. In another embodiment, the sarcopenia is any other definition of sarcopenia known in the art. Each definition of sarcopenia represents a separate embodiment of the present invention.

In another embodiment, the ADAM-associated condition is osteopenia. “Osteopenia” refers, in one embodiment, to decreased calcification or density of bone. This is a term which encompasses, in one embodiment, all skeletal systems in which such a condition is noted.

In another embodiment, the ADAM-associated condition is osteoporosis. “Osteoporosis” refers, in one embodiment, to a thinning of the bones with reduction in bone mass due to depletion of calcium and bone protein. Osteoporosis predisposes a person to fractures, which are often slow to heal and heal poorly. Unchecked osteoporosis can lead to changes in posture, physical abnormality, and decreased mobility.

“Osteoporosis” refers, in another embodiment, to a thinning of the bones with reduction in bone mass due to depletion of calcium and bone protein. In another embodiment, osteoporosis is a systemic skeletal disease, characterized by low bone mass and deterioration of bone tissue, with a consequent increase in bone fragility and susceptibility to fracture. In osteoporotic patients, bone strength is abnormal, in one embodiment, with a resulting increase in the risk of fracture. In another embodiment, osteoporosis depletes both the calcium and the protein collagen normally found in the bone, in one embodiment, resulting in either abnormal bone quality or decreased bone density. In another embodiment, bones that are affected by osteoporosis can fracture with only a minor fall or injury that normally would not cause a bone fracture. The fracture can be, in one embodiment, either in the form of cracking (as in a hip fracture) or collapsing (as in a compression fracture of the spine). The spine, hips, and wrists are common areas of osteoporosis-induced bone fractures, although fractures can also occur in other skeletal areas. Unchecked osteoporosis can lead, in another embodiment, to changes in posture, physical abnormality, and decreased mobility.

Osteoporosis and osteopenia are, in another embodiment, systemic skeletal diseases characterized by low bone mass and microarchitectural deterioration of bone tissue. “Microarchitectural deterioration” refers, in one embodiment, to thinning of the trabeculae (defined below) and the loss of inter-trabecular connections in bone. In another embodiment, “osteoporosis” is defined as having a BMD 2.5 standard deviations (SD) or more below the young adult mean. In another embodiment, “osteoporosis” is defined as having a BMC 2.5 SD or more below the young adult mean. In another embodiment, “osteoporosis” is defined as having a BMD 2.0 SD or more below the young adult mean. In another embodiment, “osteoporosis” is defined as having a BMC 2.0 SD or more below the young adult mean. In another embodiment, “osteoporosis” is defined as having a BMD 3.0 SD or more below the young adult mean. In another embodiment, “osteoporosis” is defined as having a BMC 3.0 SD or more below the young adult mean. Each definition of osteoporosis or osteopenia represents a separate embodiment of the present invention.

In another embodiment, the ADAM-associated condition is BPH. BPH is, in one embodiment, a nonmalignant enlargement of the prostate gland, and is the most common non-malignant proliferative abnormality found in any internal organ and the major cause of morbidity in the adult male. BPH occurs in over 75% of men over 50 years of age, reaching 88% prevalence by the ninth decade. BPH frequently results in a gradual squeezing of the portion of the urethra that traverses the prostate (prostatic urethra). This causes patients to experience a frequent urge to urinate because of incomplete emptying of the bladder and urgency of urination. The obstruction of urinary flow can also lead to a general lack of control over urination, including difficulty initiating urination when desired, as well as difficulty in preventing urinary flow because of the inability to empty urine from the bladder, a condition known as overflow urinary incontinence, which can lead to urinary obstruction and to urinary failure.

In another embodiment, the ADAM-associated condition is associated with an alternation in cognition and mood. The term “cognition” refers, in one embodiment, to the process of knowing, specifically the process of being aware, knowing, thinking, learning and judging. Cognition is related to the fields of psychology, linguistics, computer science, neuroscience, mathematics, ethology and philosophy. The term “mood” refers, in one embodiment, to a state of the mind. In another embodiment, “mood” refers to a tendency to anger. In another embodiment, “mood” refers to a tendency to sadness. As contemplated herein, “alterations” means, in one embodiment, any change for the positive or negative, in cognition and/or mood.

In another embodiment, the ADAM-associated condition is depression. The term “depression” refers, in one embodiment, to an illness that involves the body, mood and thoughts, that affects the way a person eats, sleeps and the way one feels about oneself, and thinks about things. The signs and symptoms of depression include loss of interest in activities, loss of appetite or overeating, loss of emotional expression, an empty mood, feelings of hopelessness, pessimism, guilt or helplessness, social withdrawal, fatigue, sleep disturbances, trouble concentrating, remembering, or making decisions, restlessness, irritability, headaches, digestive disorders or chronic pain.

In another embodiment, the ADAM-associated condition is hair loss. The term “hair loss”, medically known as alopecia, refers, in one embodiment, to baldness. In one embodiment, the baldness is male-pattern baldness. Baldness typically begins with patch hair loss on the scalp and sometimes progresses to complete baldness and even loss of body hair. Hair loss affects both males and females.

In another embodiment, the ADAM-associated condition is anemia. “Anemia” refers, in one embodiment, to the condition of having less than the normal number of red blood cells or less than the normal quantity of hemoglobin in the blood. The oxygen-carrying capacity of the blood is, therefore, decreased. Persons with anemia may feel tired and fatigue easily, appear pale, develop palpitations and become usually short of breath. Anemia is caused by four basic factors: a) hemorrhage (bleeding); b) hemolysis (excessive destruction of red blood cells); c) underproduction of red blood cells; and d) not enough normal hemoglobin. There are many forms of anemia, including aplastic anemia, benzene poisoning, Fanconi anemia, hemolytic disease of the newborn, hereditary spherocytosis, iron deficiency anemia, osteopetrosis, pernicious anemia, sickle cell disease, thalassemia, myelodysplastic syndrome, and a variety of bone marrow diseases. As contemplated herein, the SARM compounds of the present invention are useful in preventing and/or treating any one or more of the above-listed forms of anemia.

In another embodiment, the ADAM-associated condition is obesity. “Obesity” refers, in one embodiment, to the state of being well above one's normal weight. Traditionally, a person is considered to be obese if they are more than 20 percent over their ideal weight. Obesity has been more precisely defined by the National Institute of Health (NIH) as a Body to Mass Index (BMI) of 30 or above. Obesity is often multifactorial, based on both genetic and behavioral factors. Overweight due to obesity is a significant contributor to health problems. It increases the risk of developing a number of diseases including: Type 2 (adult-onset) diabetes; high blood pressure (hypertension); stroke (cerebrovascular accident or CVA); heart attack (myocardial infarction or MI); heart failure (congestive heart failure); cancer (certain forms such as cancer of the prostate and cancer of the colon and rectum); gallstones and gallbladder disease (cholecystitis); Gout and gouty arthritis; osteoarthritis (degenerative arthritis) of the knees, hips, and the lower back; sleep apnea (failure to breath normally during sleep, lowering blood oxygen); and Pickwickian syndrome (obesity, red face, underventilation and drowsiness). As contemplated herein, the term “obesity” includes any one of the above-listed obesity-related conditions and diseases. Thus the SARM compounds of the present invention are useful in preventing and/or treating obesity and any one or more of the above-listed obesity-related conditions and diseases.

In another embodiment, the ADAM-associated condition is prostate cancer. Prostate cancer is one of the most frequently occurring cancers among men in the United States, with hundreds of thousands of new cases diagnosed each year. Over sixty percent of newly diagnosed cases of prostate cancer are found to be pathologically advanced, with no cure and a dismal prognosis. One third of all men over 50 years of age have a latent form of prostate cancer that may be activated into the life-threatening clinical prostate cancer form. The frequency of latent prostatic tumors has been shown to increase substantially with each decade of life from the 50s (5.3-14%) to the 90s (40-80%). The number of people with latent prostate cancer is the same across all cultures, ethnic groups, and races, yet the frequency of clinically aggressive cancer is markedly different. This suggests that environmental factors may play a role in activating latent prostate cancer. Methods of diagnosing prostate cancer are well known in the art, and include measurement of free- and bound prostate-specific antigen (PSA) prostate exam, and prostate biopsy.

Each ADAM-related condition treated by a SARM compound represents a separate embodiment of the present invention. Each type of AR activity (agonistic, partial agonistic, and antagonistic represents a separate embodiment of the present invention. Each target tissue represents a separate embodiment of the present invention.

In one embodiment, a method of the present invention comprises administering to the subject the SARM compound. In another embodiment, the method comprises administering an analog of the SARM. In another embodiment, the method comprises administering a derivative of the SARM. In another embodiment, the method comprises administering an isomer of the SARM. In another embodiment, the method comprises administering a metabolite of the SARM. In another embodiment, the method comprises administering a pharmaceutically acceptable salt of the SARM. In another embodiment, the method comprises administering a pharmaceutical product of the SARM. In another embodiment, the method comprises administering a hydrate of the SARM. In another embodiment, the method comprises administering an N-oxide of the SARM. In another embodiment, the method comprises administering a pro-drug of the SARM. In another embodiment, the method comprises administering a polymorph of the SARM. In another embodiment, the method comprises administering a crystal of the SARM. In another embodiment, the method comprises administering any combination of a SARM, its analog, derivative, isomer, metabolite, pharmaceutically acceptable salt, pharmaceutical product, hydrate, N-oxide, pro-drug, polymorph, or crystal thereof.

The present invention further provides a class of compounds referred to as SARM compounds. Each of these compounds acts, in one embodiment, as either an androgen receptor (AR) agonist, partial agonist and/or antagonist, depending on the tissue.

In one embodiment, the SARM compound is represented by a structure of formula I:

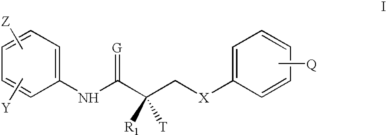

wherein
G is O or S;
X is a bond, O, CH₂, NH, Se, PR, NO or NR;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is alkyl, F, Cl, Br, I, CF₃, CN, CR₃, SnR₃, NR₂, NHCOCH₃, NHCOCF₃, NHCOR, NHCONHR, NHCOOR, OCONHR, CONHR, NHCSCH₃, NHCSCF₃, NHCSR, NHSO₂CH₃, NHSO₂R, OR, COR, OCOR, OSO₂R, SO₂R, SR, NCS, SCN, NCO, or OCN; or Q together with the benzene ring to which it is attached is a fused ring system represented by structure A, B or C: 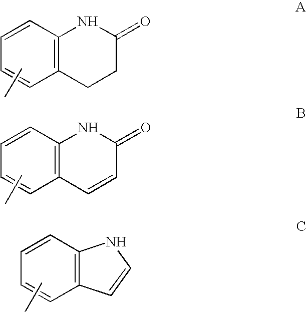
R is alkyl, haloalkyl, dihaloalkyl, trihaloalkyl, CH₂F, CHF₂, CF₃, CF₂CF₃, aryl, phenyl, F, Cl, Br, I, alkenyl or OH; and
R₁ is CH₃, CH₂F, CHF₂, CF₃, CH₂CH₃, or CF₂CF₃.

In one embodiment, the SARM compound is a compound of formula I wherein X is O. In one embodiment, the SARM compound is a compound of formula I wherein G is O. In another embodiment, the SARM compound is a compound of formula I wherein Z is NO₂. In another embodiment, the SARM compound is a compound of formula I wherein Z is CN. In another embodiment, the SARM compound is a compound of formula I wherein Y is CF₃. In another embodiment, the SARM compound is a compound of formula I wherein Q is NHCOCH₃. In another embodiment, the SARM compound is a compound of formula I wherein Q is F. In another embodiment, the SARM compound is a compound of formula I wherein Q is Cl. In another embodiment, the SARM compound is a compound of formula I wherein Q is Br In another embodiment, the SARM compound is a compound of formula I wherein T is OH. In another embodiment, the SARM compound is a compound of formula I wherein R₁ is CH₃.

The substituents Z and Y can be in any position of the ring carrying these substituents (hereinafter “A ring”). In one embodiment, the substituent Z is in the para position of the A ring. In another embodiment, the substituent Y is in the meta position of the A ring. In another embodiment, the substituent Z is in the para position of the A ring and substituent Y is in the meta position of the A ring.

The substituent Q can be in any position of the ring carrying this substituent (hereinafter “B ring”). In one embodiment, the substituent Q is in the para position of the B ring. In another embodiment, the substituent Q is NHCOCH₃ and is in the para position of the B ring. In another embodiment, the substituent Q is F and is in the para position of the B ring. In another embodiment, the substituent Q is Cl and is in the para position of the B ring. In another embodiment, the substituent Q is Br and is in the para position of the B ring.

In another embodiment, the SARM compound is represented by a structure of formula II:

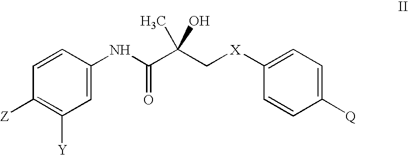

wherein
X is a bond, O, CH₂, NH, Se, PR, NO or NR;
Z is NO₂, CN, COOH, COR, NHCOR or CONHR;
Y is CF₃, F, I, Br, Cl, CN, CR₃ or SnR₃;
Q is alkyl, F, Cl, Br, I, CF₃, CN, CR₃, SnR₃, NR₂, NHCOCH₃, NHCOCF₃, NHCOR, NHCONHR, NHCOOR, OCONHR, CONHR, NHCSCH₃, NHCSCF₃, NHCSR, NHSO₂CH₃, NHSO₂R, OR, COR, OCOR, OSO₂R, SO₂R, SR, NCS, SCN, NCO, or OCN; or Q together with the benzene ring to which it is attached is a fused ring system represented by structure A, B or C: 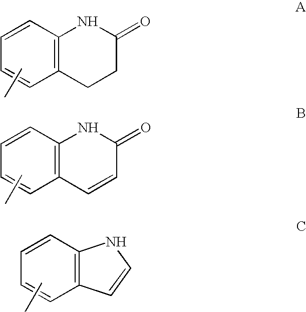
R is alkyl, haloalkyl, dihaloalkyl, trihaloalkyl, CH₂F, CHF₂, CF₃, CF₂CF₃, aryl, phenyl, F, Cl, Br, I, alkenyl or OH.

In one embodiment, the SARM compound is a compound of formula II wherein X is O. In another embodiment, the SARM compound is a compound of formula II wherein Z is NO₂. In another embodiment, the SARM compound is a compound of formula II wherein Z is CN. In another embodiment, the SARM compound is a compound of formula II wherein Y is CF₃, In another embodiment, the SARM compound is a compound of formula II wherein Q is NHCOCH₃. In another embodiment, the SARM compound is a compound of formula II wherein Q is F. In another embodiment, the SARM compound is a compound of formula H wherein Q is Cl. In another embodiment, the SARM compound is a compound of formula II wherein Q is Br.

In another embodiment, the SARM compound is represented by a structure of formula III:

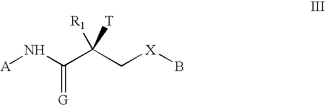

wherein
X is a bond, O, CH₂, NH, Se, PR, NO or NR;
G is O or S;

R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, F, Cl, Br, I, alkenyl or OH;
A is a ring selected from: 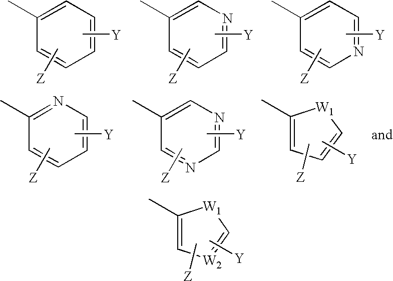
B is a ring selected from: 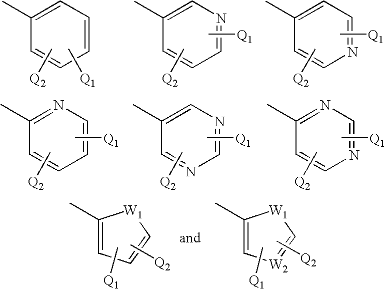
wherein
   A and B cannot simultaneously be a benzene ring;
   Z is NO₂, CN, COOH, COR, NHCOR or CONHR;
   Y is CF₃, F, I, Br, Cl, CN CR₃ or SnR₃;
   Q₁ and Q₂ are independently of each other a hydrogen, alkyl, F, Cl, Br, I, CF₃, CN, CR₃, SnR₃, NR₂, NHCOCH₃, NHCOCF₃, NHCOR, NHCONHR, NHCOOR, OCONHR, CONHR, NHCSCH₃, NHCSCF₃, NHCSR, NHSO₂CH₃, NHSO₂R, OR, COR, OCOR, OSO₂R, SO₂R, SR, NCS, SCN, NCO, OCN, 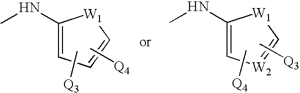
   Q₃ and Q₄ are independently of each other a hydrogen, alkyl, F, Cl, Br, I, CF₃, CN, CR₃, SnR₃, NR₂, NHCOCH₃, NHCOCF₃, NHCOR, NHCONHR, NHCOOR, OCONHR, CONHR, NHCSCH₃, NHCSCF₃, NHCSR, NHSO₂CH₃, NHSO₂R, OR, COR, OCOR, OSO₂R, SO₂R, SR, NCS, SCN, NCO or OCN;
   W₁ is O, NH, NR, NO or S; and
   W₂ is N or NO.

In one embodiment, the SARM compound is a compound of formula III wherein X is O. In another embodiment, the SARM compound is a compound of formula III wherein G is O. In another embodiment, the SARM compound is a compound of formula I wherein T is OH. In another embodiment, the SARM compound is a compound of formula III wherein R₁ is CH₃. In another embodiment, the SARM compound is a compound of formula III wherein Z is NO₂. In another embodiment, the SARM compound is a compound of formula III wherein Z is CN. In another embodiment, the SARM compound is a compound of formula III wherein Y is CF₃. In another embodiment, the SARM compound is a compound of formula III wherein Q₁ is NHCOCH₃. In another embodiment, the SARM compound is a compound of formula III wherein Q₁ is F. In another embodiment, the SARM compound is a compound of formula III wherein Q₁ is Cl. In another embodiment, the SARM compound is a compound of formula III wherein Q₁ is Br.

The substituents Z and Y can be in any position of the ring carrying these substituents (hereinafter “A ring”). In one embodiment, the substituent Z is in the para position of the A ring. In another embodiment, the substituent Y is in the meta position of the A ring. In another embodiment, the substituent Z is in the para position of the A ring and substituent Y is in the meta position of the A ring.

The substituents Q₁ and Q₂ can be in any position of the ring carrying these substituents (hereinafter “B ring”). In one embodiment, the substitutent Q₁ is in the para position of the B ring. In another embodiment, the subsituent is Q₂ is H. In another embodiment, the substitutent Q₁ is in the para position of the B ring and the subsituent is Q₂ is H. In another embodiment, the substitutent Q₁ is NHCOCH₃ and is in the para position of the B ring, and the substituent is Q₂ is H.

In another embodiment, the SARM compound is represented by a structure of formula IV:

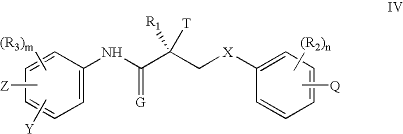

wherein
X is a bond, O, CH₂, NH, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, F, Cl, Br, I, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂ or SR;
R₃ is F, Cl, Br, I, CN, NO₂, COR, COOH, CONHR, CF₃, or SnR₃; or R₃ together with the benzene ring to which it is attached forms a fused ring system represented by the structure: 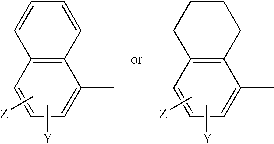
Z is NO₂, CN, COR, COOH, or CONHR;
Y is CF₃, F, Br, Cl, I, CN, or SnR₃;
Q is H, alkyl, F, Cl, Br, I, CF₃, CN, CR₃, SnR₃, NR₂, NHCOCH₃, NHCOCF₃, NHCOR, NHCONHR, NHCOOR, OCONHR, CONHR, NHCSCH₃, NHCSCF₃, NHCSR, NHSO₂CH₃, NHSO₂R, OH, OR, COR, OCOR, OSO₂R, SO₂R, SR, NCS, SCN, NCO, or OCN; or Q together with the benzene ring to which it is attached is a fused ring system represented by structure A, B or C: 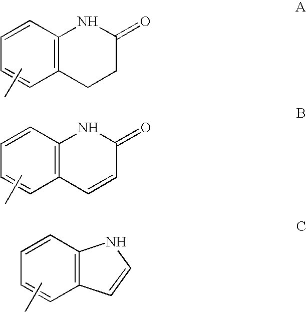
n is an integer of 1-4; and
m is an integer of 1-3.

In one embodiment, the SARM compound is a compound of formula IV wherein X is O. In another embodiment, the SARM compound is a compound of formula IV wherein G is O. In another embodiment, the SARM compound is a compound of formula IV wherein Z is NO₂. In another embodiment, the SARM compound is a compound of formula IV wherein Z is CN. In another embodiment, the SARM compound is a compound of formula IV wherein Y is CF₃. In another embodiment, the SARM compound is a compound of formula IV wherein Q is NHCOCH₃. In another embodiment, the SARM compound is a compound of formula IV wherein Q is F. In another embodiment, the SARM compound is a compound of formula IV wherein T is OH. In another embodiment, the SARM compound is a compound of formula IV wherein R₁ is CH₃. In another embodiment, the SARM compound is a compound of formula IV wherein Q is F and R₂ is CH₃. In another embodiment, the SARM compound is a compound of formula IV wherein Q is F and R₂ is Cl. In another embodiment, the SARM compound is a compound of formula IV wherein Q is Br and R₂ is Cl. In another embodiment, the SARM compound is a compound of formula IV wherein Q is F and R₂ is Br.

The substituents Z, Y and R₃ can be in any position of the ring carrying these substituents (hereinafter “A ring”). In one embodiment, the substituent Z is in the para position of the A ring. In another embodiment, the substituent Y is in the meta position of the A ring. In another embodiment, the substituent Z is in the para position of the A ring and substituent Y is in the meta position of the A ring.

The substituents Q and R₂ can be in any position of the ring carrying these substituents (hereinafter “B ring”). In one embodiment, the substitutent Q is in the para position of the B ring. In another embodiment, the substitutent Q is in the para position of the B ring. In another embodiment, the substitutent Q is NHCOCH₃ and is in the para position of the B ring.

As contemplated herein, when the integers m and n are greater than one, the substituents R₂ and R₃ are not limited to one particular substituent, and can be any combination of the substituents listed above.

In another embodiment, the SARM compound is represented by a structure of formula V:

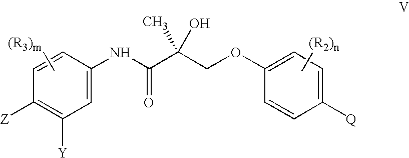

wherein
R₂ is F, Cl, Br, 1, CH₃, CF₃, OH, CN, NO₂, NHCOCH₃, NHCOCF₃, NHCOR, alkyl, arylalkyl, OR, NH₂, NHR, NR₂ or SR;
R₃ is F, Cl, Br, 1, CN, NO₂, COR, COOH, CONHR, CF₃, or SnR₃; or R₃ together with the benzene ring to which it is attached forms a fused ring system represented by the structure: 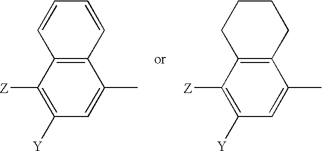
R is alkyl, haloalkyl, dihaloalkyl, trihaloalkyl, CH₂F, CHF₂, CF₃, CF₂CF₃, aryl, phenyl, F, Cl, Br, 1, alkenyl or OH;
Z is NO₂, CN, COR, COOH, or CONHR;
Y is CF₃, F, Br, Cl, I, CN, or SnR₃;
Q is H, alkyl, F, Cl, Br, I, CF₃, CN, CR₃, SnR₃, NR₂, NHCOCH₃, NHCOCF₃, NHCOR, NHCONHR, NHCOOR, OCONHR, CONHR, NHCSCH₃, NHCSCF₃, NHCSR, NHSO₂CH₃, NHSO₂R, OH, OR, COR, OCOR, OSO₂R, SO₂R, SR, NCS, SCN, NCO, or OCN; or Q together with the benzene ring to which it is attached is a fused ring system represented by structure A, B or C: 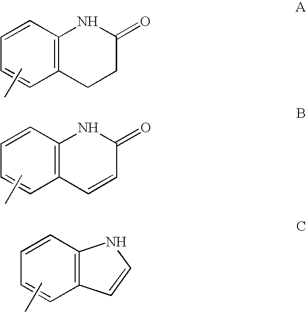
n is an integer of 1-4; and
m is an integer of 1-3.

In another embodiment, the SARM is a compound of formula V wherein Z is NO₂. In another embodiment, the SARM is a compound of formula V wherein Z is CN. In another embodiment, the SARM is a compound of formula V wherein Y is CF₃. In another embodiment, the SARM is a compound of formula V wherein Q is NHCOCH₃. In another embodiment, the SARM is a compound of formula V wherein Q is F. In another embodiment, the SARM is a compound of formula V wherein Q is F and R₂ is CH₃. In another embodiment, the SARM is a compound of formula V wherein Q is F and R₂ is Cl. In another embodiment, the SARM is a compound of formula V wherein Q is F and R₂ is Br. In another embodiment, the SARM is a compound of formula V wherein Q is Br and R₂ is Cl.

The substituents Z, Y and R₃ can be in any position of the A ring, and the substituents Q and R₂ can be in any position of B ring, as discussed above for compound IV. Furthermore, as discussed above, when the integers m and n are greater than one, the substituents R₂ and R₃ are not limited to one particular substituent, and can be any combination of the substituents listed above.

In another embodiment, the SARM compound is represented by a structure of formula VI.

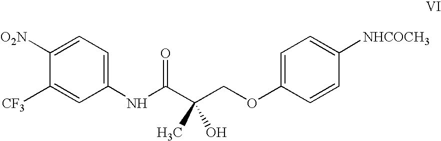

In another embodiment, the SARM compound is represented by a structure of formula VII.

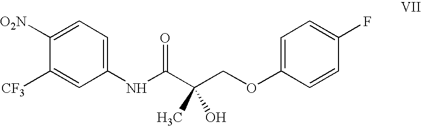

In another embodiment, the SARM compound is represented by a structure of formula VIII.

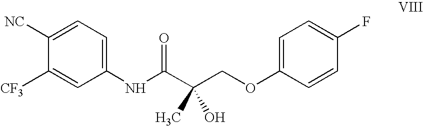

In another embodiment, the SARM compound is represented by a structure of formula IX.

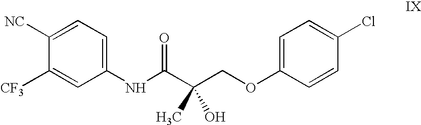

In another embodiment, the SARM compound is represented by the structure of formula X.

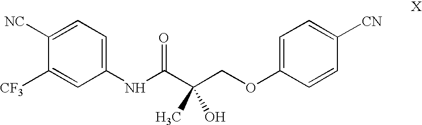

In another embodiment, the SARM compound is represented by the structure of formula XI.

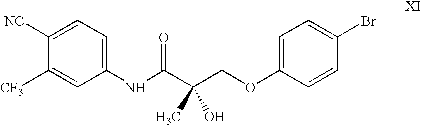

In another embodiment, the SARM compounds of the present invention may be categorized into subgroups depending on their biological activity. For example, several SARM compounds have an agonistic effect on muscle or bone. Other SARM compounds have no effect on muscle or bone. Other SARM compounds have no effect or an antagonistic effect on prostate. Other SARM compounds are able to penetrate the central nervous system (CNS). Other SARM compounds do not penetrate the CNS. Each subgroup of SARM compounds represents a separate embodiment of the present invention.

For example, one subgroup of SARM compounds has no effect on muscle and bone, and have neutral or antagonistic effect on prostate. Within this subgroup, those SARM compounds that do not penetrate the CNS are effective, in one embodiment, in treating or preventing BPH (BPH). Those SARM compounds that are able to penetrate the CNS are effective, in one embodiment, at treating or preventing sexual dysfunction.

Furthermore, another subgroup of SARM compounds has an agonistic activity on muscle and bone, and has a neutral or antagonistic effect on prostate. Within this subgroup, those SARM compounds that do not penetrate the CNS are effective, in one embodiment, at treating or preventing sarcopenia and osteopenia. Those SARM compounds that are able to penetrate the CNS are effective, in one embodiment, at treating or preventing hypogonadism, sexual dysfunction, sarcopenia and osteopenia.

In another embodiment, the present invention provides a method of treating a male subject having an ADAM-associated condition, the method comprising administering to the subject a SARM compound of the present invention.

In another embodiment, the present invention provides a method of suppressing, inhibiting or reducing an incidence of an ADAM-associated condition in a male subject, the method comprising administering to the subject a SARM compound of the present invention.

In another embodiment, the present invention provides a method of treating a male subject having a sexual dysfunction, decreased sexual libido, erectile dysfunction, hypogonadism, sarcopenia, osteopenia, osteoporosis, an alteration in cognition and mood, depression, anemia, hair loss, obesity, muscle loss, BPH, dry eye, memory loss, or prostate cancer due ADAM, the method comprising administering to the subject a SARM compound of the present invention.

In another embodiment, the SARM is an analog of one of the above SARM compounds. In another embodiment, the SARM is a derivative of one of the above SARM compounds. In another embodiment, the SARM is an isomer of one of the above SARM compounds. In another embodiment, the SARM is a metabolite of one of the above SARM compounds. In another embodiment, the SARM is a pharmaceutically acceptable salt of one of the above SARM compounds. In another embodiment, the SARM is a pharmaceutical product of one of the above SARM compounds. In another embodiment, the SARM is a hydrate of one of the above SARM compounds. In another embodiment, the SARM is an N-oxide of one of the above SARM compounds. In another embodiment, the SARM is a crystal of one of the above SARM compounds. In another embodiment, the SARM is a polymorph of one of the above SARM compounds. In another embodiment, the SARM is a pro-drug of one of the above SARM compounds. In another embodiment, the SARM is a combination of any of an analog, derivative, metabolite, isomer, pharmaceutically acceptable salt, pharmaceutical product, hydrate, N-oxide, crystal, polymorph or pro-drug of one of the above SARM compounds.

The substituent R is defined, in one embodiment, as an alkyl, haloalkyl, dihaloalkyl, trihaloalkyl, CH₂F, CHF₂, CF₃, CF₂CF₃; aryl, phenyl, F, Cl, Br, 1, alkenyl, or hydroxyl (OH).

An “alkyl” group refers, in one embodiment, to a saturated aliphatic hydrocarbon, including straight-chain, branched-chain and cyclic alkyl groups. In one embodiment, the alkyl group has 1-12 carbons. In another embodiment, the alkyl group has 1-7 carbons. In another embodiment, the alkyl group has 1-6 carbons. In another embodiment, the alkyl group has 1-4 carbons. The alkyl group may be unsubstituted or substituted by one or more groups selected from halogen (e.g. F, Cl, Br, I), hydroxy, alkoxy carbonyl, amido, alkylamido, dialkylamido, nitro, amino, alkylamino, dialkylamino, carboxyl, thio and thioalkyl.

A “haloalkyl” group refers, in one embodiment, to an alkyl group as defined above, which is substituted by one or more halogen atoms, e.g. by F, Cl, Br or I. A “halogen” refers to elements of Group VII or the periodic table, e.g. F, Cl, Br or I.

An “aryl” group refers, in one embodiment, to an aromatic group having at least one carbocyclic aromatic group or heterocyclic aromatic group, which may be unsubstituted or substituted by one or more groups selected from halogen (e.g. F, Cl, Br, I), haloalkyl, hydroxy, alkoxy carbonyl, amido, alkylamido, dialkylamido, nitro, amino, alkylamino, dialkylamino, carboxy or thio or thioalkyl. Nonlimiting examples of aryl rings are phenyl, naphthyl, pyranyl, pyrrolyl, pyrazinyl, pyrimidinyl, pyrazolyl, pyridinyl, furanyl, thiophenyl, thiazolyl, imidazolyl, isoxazolyl, and the like.

A “hydroxyl” group refers, in one embodiment, to an OH group. An “alkenyl” group refers, in one embodiment, to a group having at least one carbon-carbon double bond.

An “arylalkyl” group refers, in one embodiment, to an alkyl bound to an aryl, wherein alkyl and aryl are as defined above. An example of an aralkyl group is a benzyl group.

As defined herein, the term “isomer” includes, but is not limited to, optical isomers and analogs, structural isomers and analogs, conformational isomers and analogs, and the like.

In another embodiment, this invention encompasses the use of various optical isomers of the SARM compounds. It will be appreciated by those skilled in the art that the SARM compounds of the present invention contain at least one chiral center. Accordingly, the SARM compounds used in the methods of the present invention may exist in, and be isolated in, optically-active or racemic forms. Some compounds may also exhibit polymorphism. It is to be understood that the present invention encompasses any racemic, optically-active, polymorphic, or stereroisomeric form, or mixtures thereof, which form possesses properties useful in the treatment of obesity and related disorders as described herein. In one embodiment, the SARM compounds are the pure (R)-isomers. In another embodiment, the SARM compounds are the pure (S)-isomers. In another embodiment, the SARM compounds are a mixture of the (R)- and the (S) isomers. In another embodiment, the SARM compounds are a racemic mixture comprising an equal amount of the (R)- and the (S) isomers. It is well known in the art how to prepare optically active forms (for example, by resolution of the racemic form by recrystallization techniques, by synthesis from optically-active starting materials, by chiral synthesis, or by chromatographic separation using a chiral stationary phase).

The invention includes, in one embodiment, pharmaceutically acceptable salts of amino-substituted compounds with organic and inorganic acids, for example, citric acid and hydrochloric acid. The invention also includes N-oxides of the amino substituents of the compounds described herein Pharmaceutically acceptable salts can also be prepared from the phenolic compounds by treatment with inorganic bases, for example, sodium hydroxide. Also, esters of the phenolic compounds can be made with aliphatic and aromatic carboxylic acids, for example, acetic acid and benzoic acid esters.

This invention further includes, in one embodiment, derivatives of the SARM compounds. The term “derivatives” includes but is not limited to ether derivatives, acid derivatives, amide derivatives, ester derivatives and the like. In addition, this invention further includes hydrates of the SARM compounds. The term “hydrate” includes but is not limited to hemihydrate, monohydrate, dihydrate, trihydrate and the like.

This invention further includes, in one embodiment, metabolites of the SARM compounds. The term “metabolite” means any substance produced from another substance by metabolism or a metabolic process.

This invention further includes, in one embodiment, pharmaceutical products of the SARM compounds. The term “pharmaceutical product” means a composition suitable for pharmaceutical use (pharmaceutical composition), as defined herein.

This invention further includes, in one embodiment, pro-drugs of the SARM compounds. The term “pro-drug” means a substance which can be converted in-vivo into a biologically active agent by such reactions as hydrolysis, esterification, desterification, activation, salt formation and the like.

This invention further includes, in one embodiment, crystals of the SARM compounds. Furthermore, this invention provides polymorphs of the SARM compounds The term “crystal” means a substance in a crystalline state. The term “polymorph” refers, in one embodiment, to a particular crystalline state of a substance, having particular physical properties such as X-ray diffraction, IR spectra, melting point, and the like.

Pharmaceutical Compositions

The treatment methods of the present invention comprise, in one embodiment, administering a pharmaceutical preparation comprising the SARM compound and/or its analog, derivative, isomer, metabolite, pharmaceutically acceptable salt, pharmaceutical product, hydrate, N-oxide, pro-drug, polymorph, crystal or any combination thereof; and a pharmaceutically acceptable carrier.

As used herein, “pharmaceutical composition” means a composition comprising an “effective amount” of the active ingredient, i.e. the SARM compound, together with a pharmaceutically acceptable carrier or diluent.

An “effective amount” refers, in one embodiment, to that amount which provides a therapeutic effect for a given condition and administration regimen. An “effective amount” of the SARM compounds as used herein can be in the range of 1-500 mg/day. In one embodiment the dosage is in the range of 1-100 mg/day. In another embodiment the dosage is in the range of 100-500 mg/day. In another embodiment the dosage is in a range of 45-60 mg/day. In another embodiment the dosage is in the range of 15-25 mg/day. In another embodiment the dosage is in the range of 55-65 mg/day. In another embodiment the dosage is in the range of 45-60 mg/day. The SARM compounds can be administered daily, in single dosage forms containing the entire amount of daily dose, or can be administered daily in multiple doses such as twice daily or three times daily. The SARM compounds can also be administered intermittently, for example every other day, 3 days a week, four days a week, five days a week and the like.

The terms “treating” and “treatment” refer, in one embodiment, curative treatment. In another embodiment, the terms refer to lessening the severity of a disorder.

In another embodiment, the terms refer to lessening the frequency of outbreaks of a disorder. In another embodiment, the terms refer to remitative treatment of a disorder (i.e. treatment that causes the disorder to enter remission). The terms “reducing”, “suppressing” and “inhibiting” refer, in one embodiment, to lessening or decreasing.

The term “administering” refers, in one embodiment, to bringing a subject in contact with a SARM compound of the present invention. As used herein, administration can be accomplished in vivo, i.e. in a test tube, or in vivo, i.e. in cells or tissues of living organisms, for example humans. In one embodiment, the present invention encompasses administering the compounds of the present invention to a subject. In one embodiment, the subject is a mammalian subject. In another embodiment, the subject is a human.

The pharmaceutical compositions containing the SARM agent can be administered to a subject by any method known to a person skilled in the art, such as parenterally, paracancerally, transmucosally, transdermally, intramuscularly, intravenously, intradermally, subcutaneously, intraperitonealy, intraventricularly, intracranially, intravaginally or intratumorally.

In one embodiment, the pharmaceutical compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulstions, oils and the like. In one embodiment of the present invention, the SARM compounds are formulated in a capsule. In accordance with this embodiment, the compositions of the present invention comprise in addition to the SARM active compound and the inert carrier or diluent, a hard gelating capsule.

Further, in another embodiment, the pharmaceutical compositions are administered by intravenous, intra-arterial, or intramuscular injection of a liquid preparation. Suitable liquid formulations include solutions, suspensions, dispersions, emulsions, oils and the like. In one embodiment, the pharmaceutical compositions are administered intravenously, and are thus formulated in a form suitable for intravenous administration. In another embodiment, the pharmaceutical compositions are administered intra-arterially, and are thus formulated in a form suitable for intra-arterial administration. In another embodiment, the pharmaceutical compositions are administered intramuscularly, and are thus formulated in a form suitable for intra-muscular administration.

Further, in another embodiment, the pharmaceutical compositions are administered topically to body surfaces, and are thus formulated in a form suitable for topical administration. Suitable topical formulations include gels, ointments, creams, lotions, drops and the like. For topical administration, the SARM agents or their physiologically tolerated derivatives such as salts, esters, N-oxides, and the like are prepared and applied as solutions, suspensions, or emulsions in a physiologically acceptable diluent with or without a pharmaceutical carrier.

Further, in another embodiment, the pharmaceutical compositions are administered as a suppository, for example a rectal suppository or a urethral suppository. Further, in another embodiment, the pharmaceutical compositions are administered by subcutaneous implantation of a pellet. In a further embodiment, the pellet provides for controlled release of SARM agent over a period of time.

In another embodiment, the active compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid).

As used herein “pharmaceutically acceptable carriers or diluents” are well known to those skilled in the art. The carrier or diluent may be a solid carrier or diluent for solid formuations, a liquid carrier or diluent for liquid formulations, or mixtures thereof.

Solid carriers/diluents include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polyniethylacrylate), calcium carbonate, magnesium oxide, talc, or mixtures thereof.

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

Parenteral vehicles (for subcutaneous, intravenous, intra-arterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions Examples of oils are those of petroleum, animal, vegetable, or synthetic origin, for example, peanut oil, soybean oil, mineral oil, olive oil, sunflower oil, and fish-liver oil.

In addition, the compositions may further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents(e.g. carbomer, colloidal silicon dioxide, ethyl cellulose, guar gum), sweetners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants.

In one embodiment, the pharmaceutical compositions provided herein are controlled release compositions, i.e. compositions in which the SARM compound is released over a period of time after administration. Controlled or sustained release compositions include formulation in lipophilic depots (e.g. fatty acids, waxes, oils). In another embodiment, the composition is an immediate release composition, i.e. a composition in which the entire SARM compound is released immediately after administration.

In yet another embodiment, the pharmaceutical composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

The compositions may also include incorporation of the active material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance.

Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

Also comprehended by the invention are compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The preparation of pharmaceutical compositions that contain an active component is well understood in the art, for example by mixing, granulating, or tablet-forming processes. The active therapeutic ingredient is often mixed with excipients that are pharmaceutically acceptable and compatible with the active ingredient. For oral administration, the SARM agents or their physiologically tolerated derivatives such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. For parenteral administration, the SARM agents or their physiologically tolerated derivatives such as salts, esters, N-oxides, and the like are converted into a solution, suspension, or emulsion, if desired with the substances customary and suitable for this purpose, for example, solubilizers or other.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For use in medicine, the salts of the SARM will be pharmaceutically acceptable salts. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic: acid, oxalic acid, citric acid, tartaric acid, carbonic acid or phosphoric acid.

In one embodiment, the methods of the present invention comprise administering a SARM compound as the sole active ingredient. However, also encompassed within the scope of the present invention are methods for treating and/or preventing ADAM-associated conditions as described herein, which comprise administering the SARM compounds in combination with one or more therapeutic agents These agents include, but are not limited to: LHRH analogs, reversible anti-androgens, antiestrogens, anticancer drugs, 5-alpha reductase inhibitors, aromatase inhibitors, progestins, or agents acting through other nuclear hormone receptors.

Thus, in one embodiment, the present invention provides compositions and pharmaceutical compositions comprising a selective AR modulator compound, in combination with an LHRH analog. In another embodiment, the present invention provides compositions and pharmaceutical compositions comprising a selective AR modulator compound, in combination with a reversible anti-androgen. In another embodiment, the present invention provides compositions and pharmaceutical compositions comprising a selective AR modulator compound, in combination with an antiestrogen. In another embodiment, the present invention provides compositions and pharmaceutical compositions comprising a selective AR modulator compound, in combination with an anticancer drug. In another embodiment, the present invention provides compositions and pharmaceutical compositions comprising a selective AR modulator compound, in combination with a 5-alpha reductase inhibitor. In another embodiment, the present invention provides compositions and pharmaceutical compositions comprising a selective AR modulator compound, in combination with an aromatase inhibitor. In another embodiment, the present invention provides compositions and pharmaceutical compositions comprising a selective AR modulator compound, in combination with a progestin. In another embodiment, the present invention provides compositions and pharmaceutical compositions comprising a selective AR modulator compound, in combination with an agent acting through other nuclear hormone receptors.

### Experimental Details Section

### EXAMPLE 1

### Compound VI and Compound VII Exhibit Tissue-Specific Anabolic and Androgenic Activity in Rats of Varying Hormonal Status

### Overview

A study was performed to compare the pharmacologic effects and tissue-selectivity of 2 SARM compounds-Compound VI and Compound VII- with testosterone propionate (TP) in male rats of varying hormonal status. Male rats with normal testicular function (i.e., intact with no surgical manipulation) were included to examine the effects of these compounds on subjects with normal blood levels of testosterone. Male rats that received unilateral orchidectomy (i.e., surgical removal of one testis) were included to examine the effects of these compounds on subjects with slight androgen depletion. Male rats that received bilateral orchidectomy (i.e., surgical removal of both testes) were included to examine the effects of these compounds on androgen-deficient subjects.

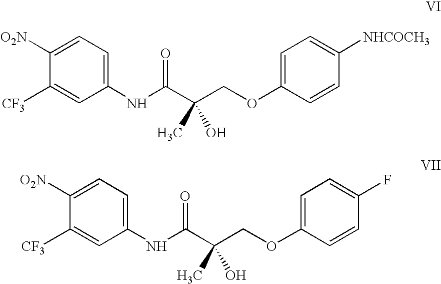

### Methods

Compound VI and Compound VII were synthesized and characterized in the laboratory of Dr. Duane Miller at the University of Tennessee, Memphis, Term. Male Sprague-Dawley rats were purchased from Harlan Biosciences (Indianapolis, Ind.). The animals were maintained on a 12-h cycle of light and dark with food and water available ad libitum. All animal studies were reviewed and approved by the Animal Care and Use Committee of The Ohio State University, and conformed to the Principles of Laboratory Animal Care (NIH publication #85-23, revised 1985). Immature male Sprague-Dawley rats weighing 187 to 214 g were randomly distributed into 9 groups of 5 animals. One day before the initiation of drug treatment, groups 4 through 6 and groups 7 through 9 received unilateral or bilateral orchidectomy, respectively, via a midline scrotal incision. Groups 1 through 3 did not undergo surgery. All drugs given to animals were freshly prepared as solutions in polyethylene glycol 300 (PEG 300) Groups 4 and 7 received treatment with vehicle alone (i.e., PEG 300). Animals in groups 3, 6, and 9 received testosterone propionate (TP, 0.5 mg/day) via implantation of subdermal osmotic pumps (Model 2002, Durect Corporation, Palo Alto, Calif.). Animals in groups 2, 5, and 8 received Compound VI or Compound VII (0.5 mg/day) via implantation of subdermal osmotic pumps. After 14 days of drug treatment, rats were weighed, anesthetized, and sacrificed. No adverse pharmacologic effects were observed upon administration of Compounds VI and VII. Blood samples were collected by venipuncture of the abdominal aorta. Plasma samples were analyzed for testosterone, Follicle Stimulating Hormone (FSH), Luteinizing Hormone (LH) and osteocalcin. Testosterone concentrations were measured by AniLytics Inc. (Gaithersburg, Md.). FSH and LH levels were measured by the National Hormone and Peptide Program (Dr A F Parlow, UCLA, CA). Plasma osteocalcin levels were determined using a commercially available rat osteocalcin EIA kit from Biomedical Technologies Inc. (Stoughton, Mass.). The ventral prostates, seminal vesicles, and levator ani muscle were removed and weighed. Osmotic pumps were also removed from animals to check for correct pump operation. The weights of all organs were normalized to body weight, and analyzed for any statistically significant differences between groups using single-factor ANOVA with the alpha value set a priori at p<0.05. The weights of prostates and seminal vesicles were used as indices for evaluation of androgenic activity, and the levator ani muscle weight was used to evaluate the anabolic activity. Statistical analyses of parameters from complete blood count or serum chemical profiling, wherever applicable, were performed by single-factor ANOVA with the alpha value set a priori at p<0.05.

Measurement of Anabolic Aid Androgenic Activity.

In vivo pharmacologic activities were determined by weight (% of intact control) of levator ani muscle, an indicator of anabolic activity. Bone formation rate, another indication of anabolic activity, was assessed by osteocalcin level measurement, as described above. Prostate and seminal vesicle were used as target tissues to indicate androgenic effects.

### Results

Plasma testosterone levels were significantly lower in castrated rats, regardless of the treatment group (Table 1), while unilateral orchidectomy led to a decrease relative to intact controls that was not statistically significant. Administration of exogenous TP raised testosterone levels relative to vehicle-treated and Compound VI treated controls in castrated rats, but not in hemi-orchidectomized animals. Compound VI treatment did not affect testosterone levels in intact, hemi-orchidectomized or castrated male rats, demonstrating that Compound VI has no significant effect on endogenous androgen production at pharmacologically relevant doses.

TABLE 1
Plasma testosterone levels (ng/ml) in different
treatment groups (n = 5).

| | | Compound VI | |
|---|---|---|---|
| | Control | (0.5 mg/day) | TP (0.5 mg/day) |
| Intact | 2.674 ± 1.476 | 1.830 ± 0.510 | 1.482 ± 0.416 |
| Hemi-orchi- | 1.740 ± 1.049 | 1.404 ± 0.810 | 2.366 ± 1.232 |
| dectomized | | | |
| Castrated | 0.036 ± 0.075^{†‡} | 0.066 ± 0.148^{†‡} | 0.258 ± 0.103*^{†‡} |

*p < 0.05 compared to control group.
^{†}p < 0.05 compared to intact group.
^{‡}p < 0.05 compared to hemi-orchidectomized group.

Plasma FSH and LH levels significantly increased in castrated animals, but not in hemi-orchidectomized animals (Tables 2 and 3), corroborating the observation that unilateral orchidectomy had no large effect on plasma testosterone levels or the pituitary hormones that regulate it. Treatment with Compound VI did not significantly affect FSH and LH levels in castrated male rats, while treatment with TP significantly decreased these levels compared to the untreated castrated animals, indicating that TP, but not Compound VI, suppresses pituitary hormone production. No treatments significantly affected FSH or LH levels in intact or hemi-orchidectomized animals.

These findings indicate that Compound VI has no effect on pituitary hormone production. Thus, SARMs have a therapeutic advantage over testosterone in patients in which suppression of FSH or LH levels is contra-indicated.

TABLE 2
Plasma FSH levels (ng/ml) in different treatment groups (n = 5).

| | | Compound VI | |
|---|---|---|---|
| | Control | (0.5 mg/day) | TP (0.5 mg/day) |
| Intact | 13.0 ± 1.3 | 14.4 ± 1.7 | 11.4 ± 1.7 |
| Hemi- | 18.0 ± 1.9^{†} | 15.2 ± 2.2 | 17.2 ± 3.3^{†} |
| orchidectomized | | | |
| Castrated | 68.6 ± 6.3^{†‡} | 69.6 ± 11.7^{†‡} | 58.0 ± 6.9*^{†‡} |

*p < 0.05 compared to control group.
^{†}p < 0.05 compared to intact group.
^{‡}p < 0.05 compared to hemi-orchidectomized group.

TABLE 3
Plasma LH levels (ng/ml) in different treatment groups (n = 5).

| | | Compound VI | |
|---|---|---|---|
| | Control | (0.5 mg/day) | TP (0.5 mg/day) |
| Intact | 0.160 ± 0.187 | 0.026 ± 0.037 | 0.168 ± 0.173 |
| Hemi-orchi- | 0.240 ± 0.268 | 0.124 ± 0.115 | 0.124 ± 0.092 |
| dectomized | | | |
| Castrated | 8.704 ± 1.709^{†‡} | 8.644 ± 2.799^{†‡} | 6.702 ± 1.513^{†‡} |

*p < 0.05 compared to control group.
^{†}p < 0.05 compared to intact group.
^{‡}p < 0.05 compared to hemi-orchidectomized group.

In order to assess the effects of these treatments on osteoporosis, plasma osteocalcin levels were measured. Osteocalcin is a specific osteoblastic marker that can be used to evaluate the endogenous bone formation rate. Treatment with Compound VI significantly increased plasma osteocalcin levels in hemi-orchidectomized and castrated animals, while TP had no effect on osteocalcin levels, as shown in Table 4. There were no significant differences in osteocalcin levels between intact, hemi-orchidectomized and castrated animals in the vehicle-treated (i.e., control) animals.

These findings demonstrated that Compound VI increases bone formation rate in male subjects with both slight and severe androgen depletion, while having no effects on testosterone, FSH, or LH levels.

TABLE 4
Plasma osteocalcin levels (ng/ml) in different
treatment groups (n = 5).

| | | Compound VI | |
|---|---|---|---|
| | Control | (0.5 mg/day) | TP (0.5 mg/day) |
| Intact | 59.403 ± 13.933 | 55.584 ± 9.715 | 74.952 ± 15.399 |
| Hemi-orchi- | 62.110 ± 14.770 | 89.804 ± 15.517*^{†} | 77.236 ± 24.418 |
| dectomized | | | |
| Castrated | 66.965 ± 11.305 | 94.215 ± 12.568*^{†} | 65.976 ± 11.213 |

*p < 0.05 compared to control group.
^{†}p < 0.05 compared to intact group.
^{‡}p < 0.05 compared to hemi-orchidectomized group.

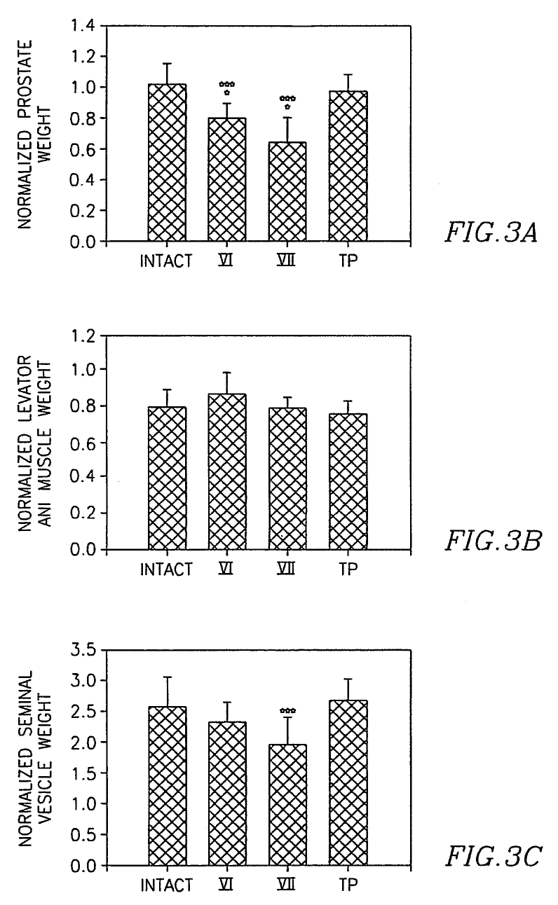

In intact animals, Compounds VI and VII decreased the size of the prostate to 79% and about 61% of control animals (Table 5; FIG. 3A). In addition, Compound VII, but not Compound VI, significantly decreased the size of the seminal vesicles (FIG. 3B). Neither Compound VI nor Compound VII significantly affected the size of the levator ani muscle (FIG. 3C).

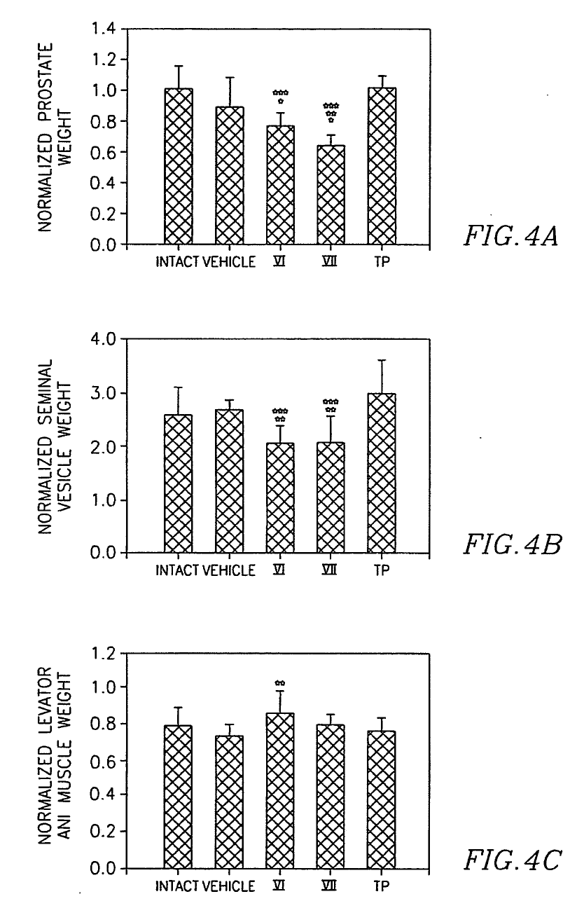

Compounds VI and VII also exhibited tissue selective pharmacologic effects in hemi-orchidectomized animals (Table 5 and FIG. 4). Compounds VI and VII decreased the size of the prostate to 75% and 60%, respectively (FIG. 4A), and each decreased the size of the seminal vesicles to 75% (FIG. 4B). Compound VI increased the size of the levator ani muscle (FIG. 4C) to 108% of untreated hemi-orchidectomized animals.

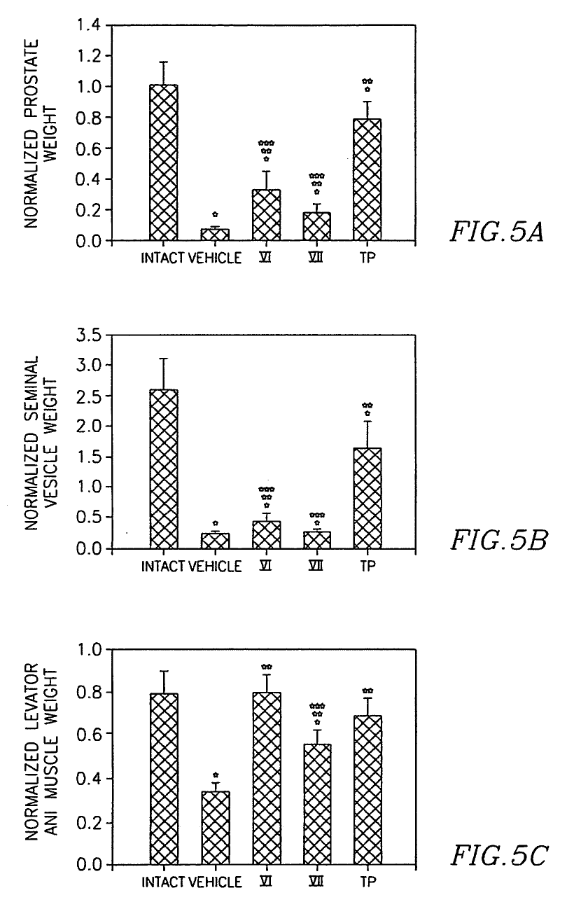

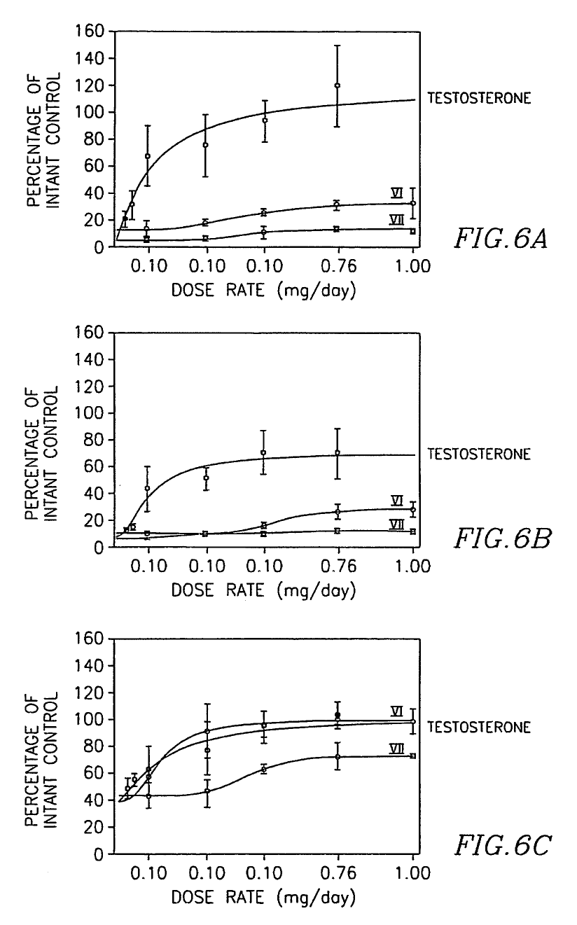

In castrated animals, as shown in Table 5 and in FIGS. 5 and 6, Compounds VI and VII increased the size of the prostate (FIG. 5A and FIG. 6A), seminal vesicles (FIG. 5B and FIG. 6B), and the levator ani muscle (FIG. 5C and FIG. 6C). In all cases of the castrated animals, differences between the SARM-treated animals and the untreated, castrated animals were statistically significant except for the seminal vesicle weight in the Compound VII-treated animals. Of note, the ratio of the anabolic activity to the androgenic activity was greater for the SARMS than for TP.

Thus, Compound VI acts as a partial AR agonist in prostate and seminal vesicles and as a full AR agonist in levator ani muscle, while Compound VII acts as a partial agonist in all tissues studied.

The findings of this Example demonstrate that (a) SARM compounds exhibit tissue selective pharmacologic effects on the AR; (b) SARM compounds exhibit anabolic activity in the absence of equally strong androgenic activity; (c) SARM compounds do not affect testosterone, FSH, or LH levels; and (d) tissue-specificity of the AR agonistic or antagonistic activity of SARM compounds is different from testosterone, and varies as well between different SARM compounds. Thus, SARM compounds are useful for a variety of ADAM-related disorders and diseases. Selection of the particular SARM will depend on the type of AR activity and target tissue of interest.

TABLE 5
Comparison of androgenic and anabolic effects of Compound VI and TP on intact,
hemi-orchidectomized and castrated rats (% of intact control, n = 5).

| Organs | | Control | Compound VI | TP |
|---|---|---|---|---|
| Prostate | Intact | 100.00 ± 13.13 | 79.41 ± 9.32*^{†} | 97.45 ± 10.82 |
| | Hemi- | 86.42 ± 19.52 | 74.69 ± 8.44*^{†} | 98.57 ± 7.98 |
| | Castrated | 7.19 ± 1.25 | 32.55 ± 11.65*^{†‡} | 76.78 ± 10.43*^{‡} |
| Seminal | Intact | 100.00 ± 18.84 | 90.54 ± 12.10 | 103.95 ± 13.23 |
| Vesicle | Hemi- | 102.93 ± 7.47 | 78.55 ± 13.58^{†‡} | 114.19 ± 23.81 |
| | Castrated | 8.97 ± 1.23 | 16.47 ± 5.21*^{†‡} | 63.48 ± 17.05*^{‡} |
| Levator | Intact | 100.00 ± 12.69 | 109.15 ± 14.68 | 95.61 ± 9.34 |
| Ani | Hemi- | 92.94 ± 7.83 | 108.10 ± 8.92^{‡} | 98.63 ± 10.47 |
| | Castrated | 42.74 ± 5.22 | 100.65 ± 0.86^{‡} | 87.27 ± 10.25^{‡} |

*p < 0.05 compared to intact control group.
^{†}p < 0.05 compared to TP of same surgical status (i.e., intact, hemi-orchidectomized, or castrate).
^{‡}p < 0.05 compared to control group of same surgical status.

### EXAMPLE 2

### Effect Of Compound VI on Myosin Heavy Chain (MHC) Subtype IIb mRNA Expression

### Methods

Expression of myosin heavy chain (MHC) subtypes was examined using RT-PCR of masseter muscle tissue of female rats by the method of Wright C et al, (Analysis of myosin heavy chain mRNA expression by RT-PCR, J Appl Physiol. 1997 October; 83(4): 1389-96). MHC is the predominant protein in skeletal muscle, is encoded by a multigene family, and is expressed in a tissue-specific and developmentally regulated manner (Adams G R et al, Time course of myosin heavy chain transitions in neonatal rats: importance of innervation and thyroid state, Am J Physiol. 1999 April; 276(4 Pt 2): R954-61). At steady state, mRNA expression parallels the pattern of MHC protein expression. Because transcription of MHC mRNA occurs in advance of MHC protein translation, and the increased sensitivity of RT-PCR compared to western blotting, rapid changes in mRNA expression can be detected and used to analyze the subtle dynamic effects on muscle metabolism.

### Results

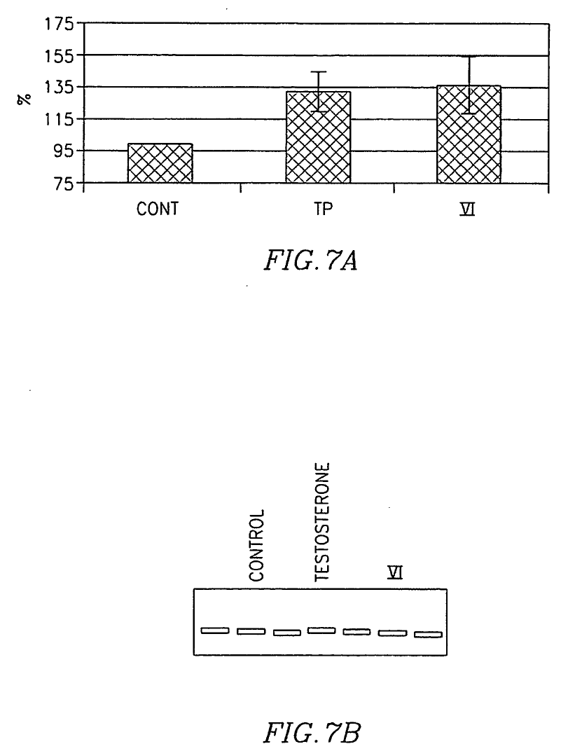

Anabolic activity of Compound VI on muscle was tested by assessing expression of MHC subtypes in skeletal muscle. MHC IIb expression level in masseter muscle of intact rats was set as the standard (100%) (FIG. 7A). TP and Compound VI both exhibited an anabolic effect on masseter muscle, increasing transcription of MHC type IIb to 133% and 137% of the untreated control (FIG. 7A). Actual untransformed PCR data is depicted in FIG. 7B.

The findings of this Example show that SARM compounds are anabolic in muscle tissue.

### EXAMPLE 3

### Effect of SARM Compounds on Bone Resorption in Rats

This study assessed the affect of SARM compounds on bone resorption in osteoporotic subjects. One hundred ten female rats were assigned to one of eleven treatment groups Groups 1-3 were intact animals, and groups 4-12 were ovariectomized on day one of the study. Groups 1 and 4 received on drug treatment, and groups 2 and 5 received dihydrotestosterone (DHT) at a dose of 1 mg/day. Groups 7-12 received Compound VI via daily subcutaneous injection at doses of 0.1, 0.3, 0.5, 0.75, 1.0, and 3 mg/day, respectively. Groups 3 and 6 received Compound VI at a dose of 1.0 mg/day together with the anti-androgen bicalutamide, to ascertain whether observed effects were mediated by the AR. All animals were treated for 120 days. Bone mineral content (BMC) was determined using dual energy x-ray absorptiometry (DEXA) on days 1, 30, 60, 90, and 120. Drug administration began immediately after oopherectomy (i.e., on the day that the operation was performed).

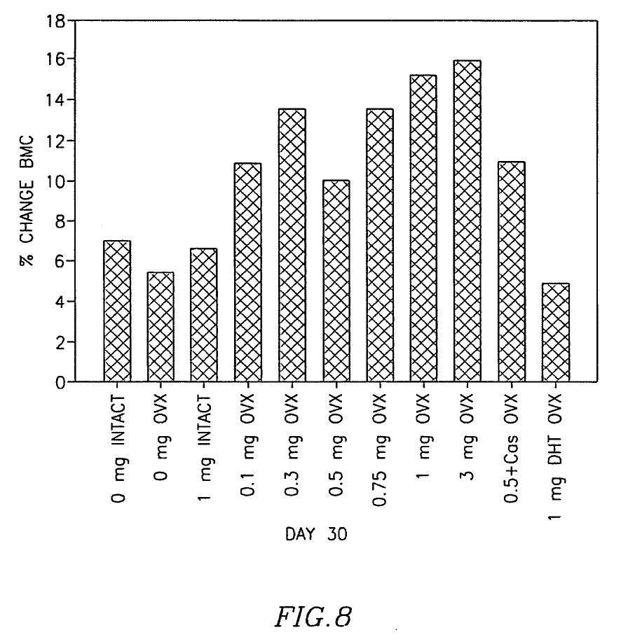

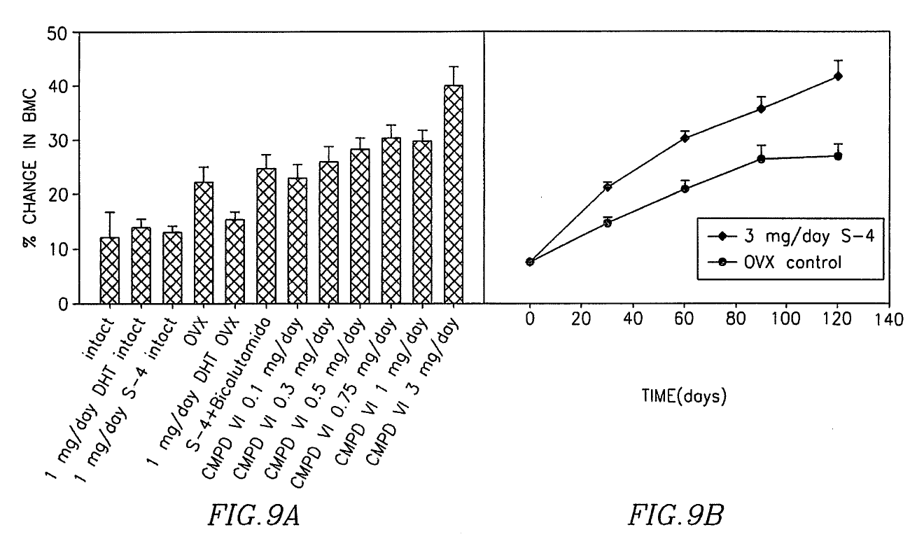

After 30 days of treatment increases in BMC were observed in Compound VI, but not DHT, treated groups (FIG. 8). As shown in FIG. 9, Compound VI increased whole body BMC in a dose-dependent and time-dependent manner, with increases of 22.9, 26.0, 28.5, 30.5, 30.0, and 40.1%, observed in groups 7-12, respectively, after 120 days (left panel), in a time-dependent fashion (right panel). By contrast, DHT increased BMC by 15%. The anti-androgen bicalutamide inhibited the effect of Compound VI in this model, indicating drug effects were mediated through the AR. Thus, Compound VI was more potent than DHT in inhibiting bone resorption.

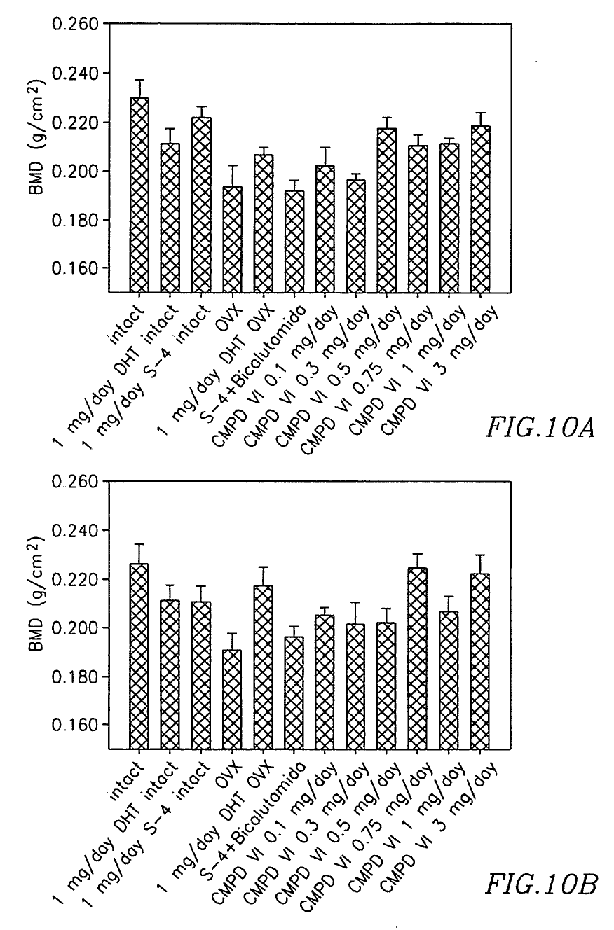

As shown in FIG. 10, Compound VI prevented bone resorption at both the L2-L4 vertebra (left panel) and the proximal femur (right panel). Bicalutamide abrogated the protective effect of Compound VI at both sites.

### Biomechanical Strength

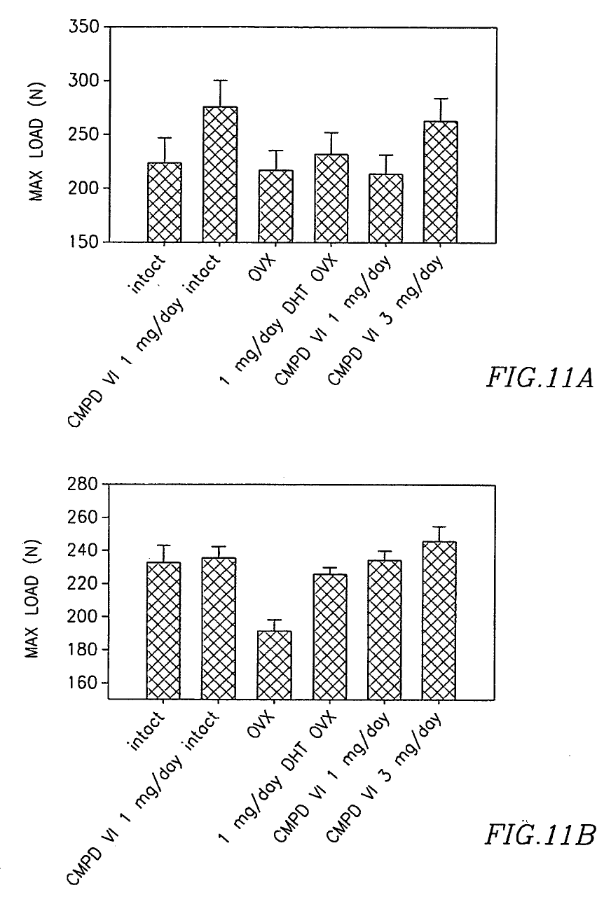

As shown in FIG. 11, Compound VI increased biomechanical strength of the L5 vertebra (left panel) and femur (right panel). Since cortical bone is largely responsible for the strength of skeletal bone, these results demonstrate that Compound VI prevented resorption of cortical bone.

### Effects on Cortical and Trabecular Bone

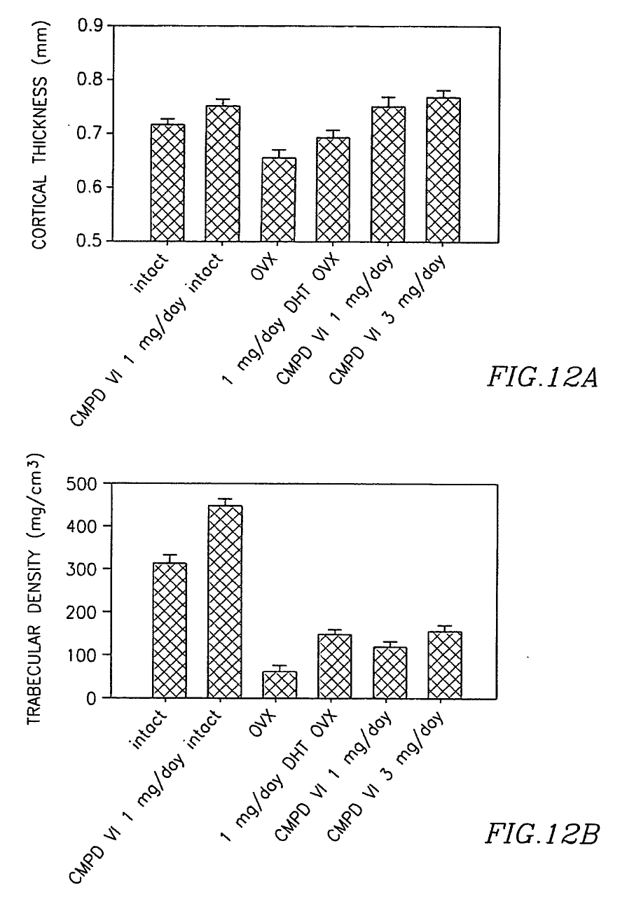

As shown in FIG. 12, left panel, Compound VI increased cortical thickness in the femoral mid-shaft relative to untreated ovarectomized animals, providing further evidence that Compound VI prevented resorption of cortical bone. In addition, Compound VI partially prevented resorption of trabecular bone, as evidenced by increased trabecular bone density in the distal femur (FIG. 12, right panel).

In summary, the finding of this Example show that SARM compounds prevent resorption of both cortical and trabecular bone. The use of multiple assays and multiple bone locations to demonstrate this finding shows that SARM compounds improve multiple aspects of bone, such as bone mass, bone quality, and biomechanical strength, and demonstrates that the improvement of bone by SARM compounds is manifest in many locations throughout the body.

In summary, the findings of the present invention demonstrate that SARM compounds (1) are anabolic in bone (both cortical and trabecular) and muscle in testosterone-depleted subjects, and subjects with slight androgen depletion; (2) have no effects on testosterone, FSH, or LH levels; (3) exhibit mixed AR agonist activity in sexual accessory glands; and (4) prevent bone resorption in testosterone-depleted subjects. The findings also demonstrate that the type and quantity of AR activity, and the tissues most effective, vary between different SARM compounds.

It will be appreciated by a person skilled in the art that the present invention is not limited by what has been particularly shown and described hereinabove. Rather, the scope of the invention is defined by the claims which follow:

What is claimed is:

1. A method of treating a male subject having an Androgen Decline in Aging Male (ADAM)-associated condition, said method comprising administering to said subject a selective androgen receptor modulator (SARM) compound represented by a structure of formula VIII: 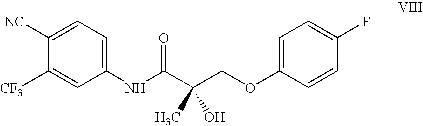
or an analog, derivative, isomer, metabolite, pharmaceutically acceptable salt, pharmaceutical product, hydrate or N-oxide, pro-drug, polymorph or crystal thereof,
thereby treating a male subject having an ADAM-associated condition.

2. The method of claim 1, further comprising administering a pharmaceutical preparation comprising said SARM compound and/or its analog, derivative, isomer, metabolite, pharmaceutically acceptable salt, pharmaceutical product, hydrate, N-oxide, pro-drug, polymorph, crystal, or any combination thereof; and a pharmaceutically acceptable carrier.

3. The method of claim 2, comprising intravenously, intra-arterially, or intramuscularly injecting to said subject said pharmaceutical preparation in liquid form; subcutaneously implanting in said subject a pellet containing said pharmaceutical preparation; orally administering to said subject said pharmaceutical preparation in a liquid or solid form; or topically applying to the skin surface of said subject said pharmaceutical preparation.

4. The method of claim 2, wherein said pharmaceutical preparation is a pellet, a tablet, a capsule, a solution, a suspension, an emulsion, an elixir, a gel, a cream, a suppository or a parenteral formulation.

5. The method of claim 1, wherein said ADAM-associated condition is sexual dysfunction, decreased sexual libido, erectile dysfunction, hypogonadism, sarcopenia, osteopenia, osteoporosis, an alteration in cognition and mood, depression, anemia, hair loss, obesity, muscle loss, dry eye, memory loss, benign prostate hyperplasia, prostate cancer, or any combination thereof.

6. The method of claim 1, wherein said male subject is an aging male subject.

7. A method of treating a male subject having an Androgen Decline in Aging Male (ADAM)-associated condition, said method comprising administering to said subject a selective androgen receptor modulator (SARM) compound represented by a structure of formula IX: 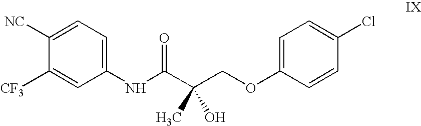
or an analog, derivative, isomer, metabolite, pharmaceutically acceptable salt, pharmaceutical product, hydrate or N-oxide, pro-drug, polymorph or crystal thereof,
thereby treating a male subject having an ADAM-associated condition.

8. The method of claim 7, further comprising administering a pharmaceutical preparation comprising said SARM compound and/or its analog, derivative, isomer, metabolite, pharmaceutically acceptable salt, pharmaceutical product, hydrate, N-oxide, pro-drug, polymorph, crystal, or any combination thereof; and a pharmaceutically acceptable carrier.

9. The method of claim 8, comprising intravenously, intra-arterially, or intramuscularly injecting to said subject said pharmaceutical preparation in liquid form; subcutaneously implanting in said subject a pellet containing said pharmaceutical preparation; orally administering to said subject said pharmaceutical preparation in a liquid or solid form; or topically applying to the skin surface of said subject said pharmaceutical preparation.

10. The method of claim 8, wherein said pharmaceutical preparation is a pellet, a tablet, a capsule, a solution, a suspension, an emulsion, an elixir, a gel, a cream, a suppository or a parenteral formulation.

11. The method of claim 7, wherein said ADAM-associated condition is sexual dysfunction, decreased sexual libido, erectile dysfunction, hypogonadism, sarcopenia, osteopenia, osteoporosis, an alteration in cognition and mood, depression, anemia, hair loss, obesity, muscle loss, dry eye, memory loss, benign prostate hyperplasia, prostate cancer, or any combination thereof.

12. The method of claim 7, wherein said male subject is an aging male subject.

13. A method of treating a male subject having an Androgen Decline in Aging Male (ADAM)-associated condition, said method comprising administering to said subject a selective androgen receptor modulator (SARM) compound represented by a structure of formula X: 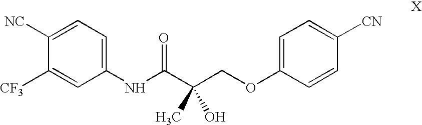
or an analog, derivative, isomer, metabolite, pharmaceutically acceptable salt, pharmaceutical product, hydrate or N-oxide, pro-drug, polymorph or crystal thereof,
thereby treating a male subject having an ADAM-associated condition.

14. The method of claim 13, further comprising administering a pharmaceutical preparation comprising said SARM compound and/or its analog, derivative, isomer, metabolite, pharmaceutically acceptable salt, pharmaceutical product, hydrate, N-oxide, pro-drug, polymorph, crystal, or any combination thereof; and a pharmaceutically acceptable carrier.

15. The method of claim 14, comprising intravenously, intra-arterially, or intramuscularly injecting to said subject said pharmaceutical preparation in liquid form; subcutaneously implanting in said subject a pellet containing said pharmaceutical preparation; orally administering to said subject said pharmaceutical preparation in a liquid or solid form; or topically applying to the skin surface of said subject said pharmaceutical preparation.

16. The method of claim 14, wherein said pharmaceutical preparation is a pellet, a tablet, a capsule, a solution, a suspension, an emulsion, an elixir, a gel, a cream, a suppository or a parenteral formulation.

17. The method of claim 13, wherein said ADAM-associated condition is sexual dysfunction, decreased sexual libido, erectile dysfunction, hypogonadism, sarcopenia, osteopenia, osteoporosis, an alteration in cognition and mood, depression, anemia, hair loss, obesity, muscle loss, dry eye, memory loss, benign prostate hyperplasia, prostate cancer, or any combination thereof.

18. The method of claim 13, wherein said male subject is an aging male subject.

19. A method of suppressing, inhibiting or reducing an incidence of an Androgen Decline in Aging Male (ADAM)-associated condition in a male subject, said method comprising administering to said subject a selective androgen receptor modulator (SARM) compound represented by a structure of formula VIII: 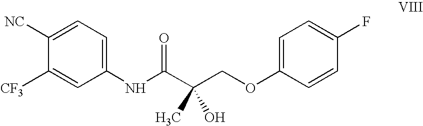
or an analog, derivative, isomer, metabolite, pharmaceutically acceptable salt, pharmaceutical product, hydrate or N-oxide, pro-drug, polymorph or crystal thereof,
thereby suppressing, inhibiting or reducing an incidence of an ADAM-associated condition in a male subject.

20. The method of claim 19, further comprising administering a pharmaceutical preparation comprising said SARM compound and/or its analog, derivative, isomer, metabolite, pharmaceutically acceptable salt, pharmaceutical product, hydrate, N-oxide, pro-drug, polymorph, crystal, or any combination thereof; and a pharmaceutically acceptable carrier.

21. The method of claim 20, comprising intravenously, intra-arterially, or intramuscularly injecting to said subject said pharmaceutical preparation in liquid form; subcutaneously implanting in said subject a pellet containing said pharmaceutical preparation; orally administering to said subject said pharmaceutical preparation in a liquid or solid form; or topically applying to the skin surface of said subject said pharmaceutical preparation.

22. The method of claim 20, wherein said pharmaceutical preparation is a pellet, a tablet, a capsule, a solution, a suspension, an emulsion, an elixir, a gel, a cream, a suppository or a parenteral formulation.

23. The method of claim 19, wherein said ADAM-associated condition is sexual dysfunction, decreased sexual libido, erectile dysfunction, hypogonadism, sarcopenia, osteopenia, osteoporosis, an alteration in cognition and mood, depression, anemia, hair loss, obesity, muscle loss, dry eye, memory loss, benign prostate hyperplasia, prostate cancer, or any combination thereof.

24. The method of claim 19, wherein said male subject is an aging male subject.

25. A method of suppressing, inhibiting or reducing an incidence of an Androgen Decline in Aging Male (ADAM)-associated condition in a male subject, said method comprising administering to said subject a selective androgen receptor modulator (SARM) compound represented by a structure of formula IX: 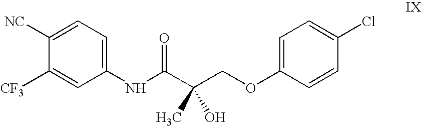
or an analog, derivative, isomer, metabolite, pharmaceutically acceptable salt, pharmaceutical product, hydrate or N-oxide, pro-drug, polymorph or crystal thereof,
thereby suppressing, inhibiting or reducing an incidence of an ADAM-associated condition in a male subject.

26. The method of claim 25, further comprising administering a pharmaceutical preparation comprising said SARM compound and/or its analog, derivative, isomer, metabolite, pharmaceutically acceptable salt, pharmaceutical product, hydrate, N-oxide, pro-drug, polymorph, crystal, or any combination thereof; and a pharmaceutically acceptable carrier.

27. The method of claim 26, comprising intravenously, intra-arterially, or intramuscularly injecting to said subject said pharmaceutical preparation in liquid form; subcutaneously implanting in said subject a pellet containing said pharmaceutical preparation; orally administering to said subject said pharmaceutical preparation in a liquid or solid form; or topically applying to the skin surface of said subject said pharmaceutical preparation.

28. The method of claim 26, wherein said pharmaceutical preparation is a pellet, a tablet, a capsule, a solution, a suspension, an emulsion, an elixir, a gel, a cream, a suppository or a parenteral formulation.

29. The method of claim 25, wherein said ADAM-associated condition is sexual dysfunction, decreased sexual libido, erectile dysfunction, hypogonadism, sarcopenia, osteopenia, osteoporosis, an alteration in cognition and mood, depression, anemia, hair loss, obesity, muscle loss, dry eye, memory loss, benign prostate hyperplasia, prostate cancer, or any combination thereof.

30. The method of claim 25, wherein said male subject is an aging male subject.

31. A method of suppressing, inhibiting or reducing an incidence of an Androgen Decline in Aging Male (ADAM)-associated condition in a male subject, said method comprising administering to said subject a selective androgen receptor modulator (SARM) compound represented by a structure of formula X: 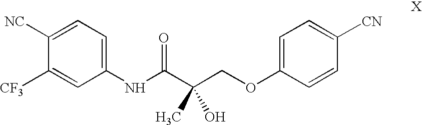
or an analog, derivative, isomer, metabolite, pharmaceutically acceptable salt, pharmaceutical product, hydrate or N-oxide, pro-drug, polymorph or crystal thereof,
thereby suppressing, inhibiting or reducing an incidence of an ADAM-associated condition in a male subject.

32. The method of claim 31, further comprising administering a pharmaceutical preparation comprising said SARM compound and/or its analog, derivative, isomer, metabolite, pharmaceutically acceptable salt, pharmaceutical product, hydrate, N-oxide, pro-drug, polymorph, crystal, or any combination thereof; and a pharmaceutically acceptable carrier.

33. The method of claim 32, comprising intravenously, intra-arterially, or intramuscularly injecting to said subject said pharmaceutical preparation in liquid form; subcutaneously implanting in said subject a pellet containing said pharmaceutical preparation; orally administering to said subject said pharmaceutical preparation in a liquid or solid form; or topically applying to the skin surface of said subject said pharmaceutical preparation.

34. The method of claim 32, wherein said pharmaceutical preparation is a pellet, a tablet, a capsule, a solution, a suspension, an emulsion, an elixir, a gel, a cream, a suppository or a parenteral formulation.

35. The method of claim 31, wherein said ADAM-associated condition is sexual dysfunction, decreased sexual libido, erectile dysfunction, hypogonadism, sarcopenia, osteopenia, osteoporosis, an alteration in cognition and mood, depression, anemia, hair loss, obesity, muscle loss, dry eye, memory loss, benign prostate hyperplasia, prostate cancer, or any combination thereof.

36. The method of claim 31, wherein said male subject is an aging male subject.

37. A method of treating a male subject having a sexual dysfunction, decreased sexual libido, erectile dysfunction, hypogonadism, sarcopenia, osteopenia, osteoporosis, an alteration in cognition and mood, depression, anemia, hair loss, obesity, muscle loss, dry eye, memory loss, benign prostate hyperplasia or prostate cancer due to Androgen Decline in an Aging Male (ADAM), said method comprising administering to said subject a selective androgen receptor modulator (SARM) compound represented by a structure of formula VIII: 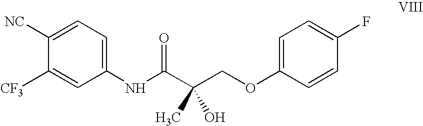
or an analog, derivative, isomer, metabolite, pharmaceutically acceptable salt, pharmaceutical product, hydrate or N-oxide, pro-drug, polymorph or crystal thereof,
thereby treating a male subject having a sexual dysfunction, decreased sexual libido, erectile dysfunction, hypogonadism, sarcopenia, osteopenia, osteoporosis, an alteration in cognition and mood, depression, anemia, hair loss, obesity, muscle loss, dry eye, memory loss, benign prostate hyperplasia or prostate cancer due ADAM.

38. The method of claim 37, further comprising administering a pharmaceutical preparation comprising said SARM compound and/or its analog, derivative, isomer, metabolite, pharmaceutically acceptable salt, pharmaceutical product, hydrate, N-oxide, pro-drug, polymorph, crystal, or any combination thereof; and a pharmaceutically acceptable carrier.

39. The method of claim 38, comprising intravenously, intra-arterially, or intramuscularly injecting to said subject said pharmaceutical preparation in liquid form; subcutaneously implanting in said subject a pellet containing said pharmaceutical preparation; orally administering to said subject said pharmaceutical preparation in a liquid or solid form; or topically applying to the skin surface of said subject said pharmaceutical preparation.

40. The method of claim 38, wherein said pharmaceutical preparation is a pellet, a tablet, a capsule, a solution, a suspension, an emulsion, an elixir, a gel, a cream, a suppository or a parenteral formulation.

41. The method of claim 38, wherein said male subject is an aging male subject.

42. A method of treating a male subject having a sexual dysfunction, decreased sexual libido, erectile dysfunction, hypogonadism, sarcopenia, osteopenia, osteoporosis, an alteration in cognition and mood, depression, anemia, hair loss, obesity, muscle loss, dry eye, memory loss, benign prostate hyperplasia or prostate cancer due to Androgen Decline in an Aging Male (ADAM), said method comprising administering to said subject a selective androgen receptor modulator (SARM) compound represented by a structure of formula IX: 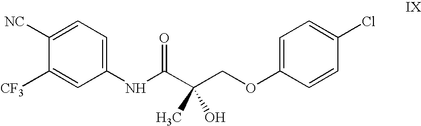
or an analog, derivative, isomer, metabolite, pharmaceutically acceptable salt, pharmaceutical product, hydrate or N-oxide, pro-drug, polymorph or crystal thereof,
thereby treating a male subject having a sexual dysfunction, decreased sexual libido, erectile dysfunction, hypogonadism, sarcopenia, osteopenia, osteoporosis, an alteration in cognition and mood, depression, anemia, hair loss, obesity, muscle loss, dry eye, memory loss, benign prostate hyperplasia or prostate cancer due ADAM.

43. The method of claim 42, further comprising administering a pharmaceutical preparation comprising said SARM compound and/or its analog, derivative, isomer, metabolite, pharmaceutically acceptable salt, pharmaceutical product, hydrate, N-oxide, pro-drug, polymorph, crystal, or any combination thereof; and a pharmaceutically acceptable carrier.

44. The method of claim 43, comprising intravenously, intra-arterially, or intramuscularly injecting to said subject said pharmaceutical preparation in liquid form; subcutaneously implanting in said subject a pellet containing said pharmaceutical preparation; orally administering to said subject said pharmaceutical preparation in a liquid or solid form; or topically applying to the skin surface of said subject said pharmaceutical preparation.

45. The method of claim 43, wherein said pharmaceutical preparation is a pellet, a tablet, a capsule, a solution, a suspension, an emulsion, an elixir, a gel, a cream, a suppository or a parenteral formulation.

46. The method of claim 42, wherein said male subject is an aging male subject.

47. A method of treating a male subject having a sexual dysfunction, decreased sexual libido, erectile dysfunction, hypogonadism, sarcopenia, osteopenia, osteoporosis, an alteration in cognition and mood, depression, anemia, hair loss, obesity, muscle loss, dry eye, memory loss, benign prostate hyperplasia or prostate cancer due to Androgen Decline in an Aging Male (ADAM), said method comprising administering to said subject a selective androgen receptor modulator (SARM) compound represented by a structure of formula X: 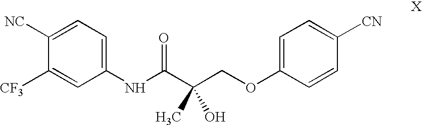
or an analog, derivative, isomer, metabolite, pharmaceutically acceptable salt, pharmaceutical product, hydrate or N-oxide, pro-drug, polymorph or crystal thereof,
thereby treating a male subject having a sexual dysfunction, decreased sexual libido, erectile dysfunction, hypogonadism, sarcopenia, osteopenia, osteoporosis, an alteration in cognition and mood, depression, anemia, hair loss, obesity, muscle loss, dry eye, memory loss, benign prostate hyperplasia or prostate cancer due ADAM.

48. The method of claim 47, further comprising administering a pharmaceutical preparation comprising said SARM compound and/or its analog, derivative, isomer, metabolite, pharmaceutically acceptable salt, pharmaceutical product, hydrate, N-oxide, pro-drug, polymorph, crystal, or any combination thereof; and a pharmaceutically acceptable carrier.

49. The method of claim 48, comprising intravenously, intra-arterially, or intramuscularly injecting to said subject said pharmaceutical preparation in liquid form; subcutaneously implanting in said subject a pellet containing said pharmaceutical preparation; orally administering to said subject said pharmaceutical preparation in a liquid or solid form; or topically applying to the skin surface of said subject said pharmaceutical preparation.

50. The method of claim 48, wherein said pharmaceutical preparation is a pellet, a tablet, a capsule, a solution, a suspension, an emulsion, an elixir, a gel, a cream, a suppository or a parenteral formulation.

51. The method of claim 47, wherein said male subject is an aging male subject.
